(19) United States
(12) Patent Application Publication
(10) Pub. No.: US 2021/0093695 A1
(43) Pub. Date: Apr. 1, 2021

(54) Method

(71) Applicant: APITOPE TECHNOLOGY (BRISTOL) LIMITED, Chepstow Gwent (GB)

(72) Inventors: Keith Martin, Chepstow Gwent (GB); Liselotta Jansson, Chepstow Gwent (GB)

(21) Appl. No.: 16/634,899

(22) PCT Filed: Aug. 14, 2018
(86) PCT No.: PCT/GB2018/052304

§ 371 Date: Jan. 29, 2020

(30) Foreign Application Priority Data
Aug. 14, 2017 (GB) 1713035.2
Aug. 14, 2017 (GB) 1713036.0
Aug. 14, 2017 (GB) 1713037.8

(51) Int. Cl. A61K 38/17; A61P 25/28

(52) U.S. Cl. CPC A61K 38/1709; A61P 25/28

(57) ABSTRACT

The present invention relates to peptides derivable from a component of myelin, namely myelin basic protein (MBP), myelin oligodendrocyte glycoprotein (MOG) or myelin proteolipid protein (PLP), for use in the treatment or prevention of impaired cognition, particularly in subjects with multiple sclerosis (MS), dementia and/or demyelination in a subject. The peptides may be used in methods of treating subjects with impaired cognition, or preventing impaired cognition, particularly in subjects with MS, treating subjects with dementia, or preventing dementia, and/or treating demyelination in a subject, or preventing demyelination in a subject.

### FIELD OF THE INVENTION

The present invention relates to peptides derivable from a component of myelin, namely myelin basic protein (MBP), myelin oligodendrocyte glycoprotein (MOG) or myelin proteolipid protein (PLP), for use in the treatment or prevention of impaired cognition, particularly in subjects with multiple sclerosis (MS), dementia and/or demyelination in a subject. The peptides may be used in methods of treating subjects with impaired cognition, or preventing impaired cognition, particularly in subjects with MS, treating subjects with dementia, or preventing dementia, and/or treating demyelination in a subject, or preventing demyelination in a subject.

### BACKGROUND TO THE INVENTION

A neuron, or neurone or nerve cell is a cell that processes and transmits information through electrical and chemical signals. Neurons are major components of the brain and spinal cord of the central nervous system (CNS), and of the autonomic ganglia of the peripheral nervous system. Neurons are capable of electrical excitation. Neurons can connect to each other to form neural networks, and signals between neurons occur via synapses.

A typical neuron consists of a cell body (soma), dendrites, and an axon. The term neurite is used to describe either a dendrite or an axon, particularly in its undifferentiated stage. Dendrites are thin structures that arise from the cell body, often extending for hundreds of micrometres and branching multiple times, giving rise to a complex “dendritic tree”. An axon (also called a nerve fiber when myelinated) is a special cellular extension (process) that arises from the cell body at the axon hillock and travels for a distance, as far as 1 meter in humans or even more in other species.

Myelin is a fatty white substance that surrounds the axon of some nerve cells, forming an electrically insulating layer known as the myelin sheath. It is essential for the proper functioning of the nervous system.

The production of the myelin sheath is due to myelination or myelinogenesis. In humans, myelination begins early in the 3rd trimester, although little myelin exists in the brain at the time of birth. During infancy, myelination occurs quickly, leading to a child's fast development, including crawling and walking in the first year. Myelination continues through the adolescent stage of life.

Demyelination is the act of demyelinating, or the loss of the myelin sheath insulating the nerves, and is the hallmark of some neurodegenerative diseases. When myelin degrades, conduction of signals along the nerve can be impaired or lost, and the nerve eventually withers. This leads to certain neurodegenerative disorders such as multiple sclerosis and chronic inflammatory demyelinating polyneuropathy.

Demyelination may result from immunological attack on neurones.

Multiple sclerosis (MS) is a chronic degenerative disease affecting the central nervous system, characterized by demyelination of nerve axons. Cognitive changes are a common symptom of MS.

Demyelination may also be involved in dementia, and conditions such as Alzheimer's disease and Parkinson's disease.

There is a need in the art for therapeutic options for treating or preventing cognitive impairment (particularly in subjects with MS), dementia and/or demyelination in a subject.

### BRIEF DESCRIPTION

The present inventors have shown that certain peptides deriving from myelin that facilitate immunological tolerance to myelin lead to upregulation of anti-inflammatory cytokines, which is commensurate with upregulation of regulatory T cells. The present Examples show that anti-inflammatory cytokines are increased with administration of the myelin-derived peptides and pro-inflammatory cytokines are reduced at the central nervous system. The Examples also show that myelin-derived peptides reduce central nervous system inflammation, and that T and B cell infiltration was reduced with administration of the peptides. Furthermore, the Examples show that administration of certain peptides deriving from myelin lead to a significant improvement in cognitive impairment in subjects with multiple sclerosis.

As shown in FIG. 1, it is proposed that activated effector immune cells cause damage to myelin and neurons. An upregulation or activation of regulatory T cells (commensurate with an increase in anti-inflammatory cytokines) is proposed to reduce the response of immune effector cells, and thereby decrease damage to myelin.

Dombrowski et al. (Nature Neuroscience 2017, 20: 674-680) have reported that regulatory T cells promote myelin regeneration in the central nervous system. The authors found that regulatory T cells promote oligodendrocyte differentiation and remyelination. Treg-deficient mice exhibited substantially impaired remyelination and oligodendrocyte differentiation. The findings revealed a new regenerative function of Treg in the CNS.

Dansokho et al. (Brain 2016, 139:1237-1251) also report that regulatory T cells delay disease progression in Alzheimer-like pathology. The authors suggest that regulatory T cells play a beneficial role in the pathophysiology of Alzheimer's disease.

In addition, Späni et al. (Acta Neuropathalogica Communications 2015, 3:71) describe that in Alzheimer's disease, accumulation and pathological aggregation of amyloid β-peptide is accompanied by the induction of immune responses. The experimental work carried out by the authors revealed reduced amyloid β-peptide pathology and reduced brain amyloid β-peptide levels in mice lacking functional adaptive immune cells.

Zhan et al. (J Alzheimer's Dis. 2015, 44:1213-1229) have demonstrated that myelin and axons in grey matter are damaged in Alzheimer's disease brains. There was evidence of degraded myelin basic protein in AD grey matter and AD neurones it was concluded that damaged axons could be a source of amyloid β precursor protein, and that MBP and degraded MBP associate with myeloid plaques and amyloid β precursor protein, and that these molecules may be associated with the formation of amyloid plaques.

Laurent et al. (Brain 2017, 140:184-200) demonstrate that hippocampal tau pathology is associated with chemokine production and parenchymal T cell infiltration, and suggested a role for immunity in tau-triggered cognitive defects in Alzheimer's disease.

Bryson and Lynch (Curr. Opin. Pharmacol. 2016, 26:67-73) have also linked T cells to Alzheimer's disease.

Furthermore, in respect of Parkinson's disease, it is described in several papers that antibody titre is increased to myelin proteins confirming an ongoing inflammatory neurodegenerative process of myelin sheaths (see, for example, Papuć E, et al., Ann Agric Environ Med. 2016; 23(2), Papuć E, et al, Neurosci Lett, Apr. 30, 2014). It has been suggested that this antibody increase is associated with dementia in Parkinson's disease (see Maetzler et al., J Alzheimer, 26, 2011). Gagne and Power (Neurology 2010, 74:995-1002) also suggest a possible neuroinflammatory pathway in Parkinson's disease pathogenesis. Ding et al. (Eur. Rev. Med. Pharmacol. Sci. 2015, 19:2275-2281) reviewed neurodegeneration and cognition in Parkinson's disease.

It is therefore proposed that the increased or activated regulatory T cell response facilitated by the myelin-derived peptides as described herein will be beneficial in the treatment or prevention of cognitive impairment, dementia and/or demyelination.

As such, the peptides deriving from myelin are now proposed as a therapeutic option in the treatment or prevention of cognitive impairment, dementia and/or demyelination.

As such, the invention provides a method for treating or preventing cognitive impairment, dementia and/or demyelination in a subject comprising administering to the subject a peptide derived or derivable from a component of myelin selected from myelin basic protein (MBP), myelin oligodendrocyte glycoprotein (MOG) and myelin proteolipid protein (PLP).

The present invention represents the first time that such peptides derived from myelin have been proposed for use in treating cognitive impairment, dementia and/or demyelination, and represents an important treatment option in treating or preventing cognitive impairment, dementia and/or demyelination, and promoting remyelination.

In another aspect the invention provides a peptide derived or derivable from a component of myelin selected from myelin basic protein (MBP), myelin oligodendrocyte glycoprotein (MOG) and myelin proteolipid protein (PLP) for use in treating or preventing impaired cognition, dementia and/or demyelination in a subject.

In one aspect is provided use of a peptide derived or derivable from a component of myelin selected from myelin basic protein (MBP), myelin oligodendrocyte glycoprotein (MOG) and myelin proteolipid protein (PLP) in the manufacture of a medicament for use in the treatment or prevention of impaired cognition, dementia and/or demyelination.

In one aspect is provided use of a peptide derived or derivable from a component of myelin selected from myelin basic protein (MBP), myelin oligodendrocyte glycoprotein (MOG) and myelin proteolipid protein (PLP) for treating or preventing impaired cognition, dementia and/or demyelination in a subject.

As discussed herein, the peptides for use according to the invention have been demonstrated previously to lead to immunological tolerance in respect of the myelin components MBP, MOG or PLP.

Cognitive impairment, dementia and/or demyelination may result from neurodegeneration due to an immunological attack on neurones. In one aspect, cognitive impairment, dementia and/or demyelination may be as a result of Alzheimer's disease or Parkinson's disease.

In one aspect the subject has a demyelinating disease. A demyelinating disease may include any condition that results in neurodegeneration resulting from an immunological attack on neurones.

In one aspect, the demyelinating disease may include Alzheimer's disease or Parkinson's disease.

In one aspect of the invention the demyelinating disease is multiple sclerosis.

In one aspect the subject is a human subject.

In one aspect the invention provides a method for promoting remyelination of a neuron, for example by contacting said neuron with a peptide as described herein. The neuron may have undergone demyelination. In one aspect the method is an in vitro method.

The invention also provides a kit for treating or preventing cognitive impairment, dementia and/or demyelination in a subject wherein said kit comprises a peptide derived or derivable from a component of myelin selected from myelin basic protein (MBP), myelin oligodendrocyte glycoprotein (MOG) and myelin proteolipid protein (PLP). The peptides of the kit may be for simultaneous, separate or sequential administration.

The peptide according to any aspect may be in the form of a composition, for example a pharmaceutical composition.

In one aspect the peptide is selected from SEQ ID Nos. 1, 2, 3 and 4. In one aspect of the invention as described herein, peptides of SEQ ID Nos. 1, 2, 3 and 4 are administered to said subject.

As such, in one aspect the composition comprises MBP30-44, MBP83-99, MBP131-145 and MBP140-154 (the combination of which is also referred to herein as “ATX-MS-1467”). In one aspect the peptides in the composition consist or consist essentially of MBP30-44, MBP83-99, MBP131-145 and MBP140-154.

In one aspect the composition does not include any other peptides in addition to MBP30-44, MBP83-99, MBP131-145 and MBP140-154.

In a preferred embodiment the invention provides a method for treating cognitive impairment in a subject who has multiple sclerosis comprising administering to the subject MBP30-44, MBP83-99, MBP131-145 and MBP140-154 peptides, preferably a composition comprising MBP30-44, MBP83-99, MBP131-145 and MBP140-154 peptides (SEQ ID Nos. 1, 2, 3 and 4).

In a preferred embodiment the invention provides a method for treating dementia in a subject comprising administering to the subject MBP30-44, MBP83-99, MBP131-145 and MBP140-154 peptides, preferably a composition comprising MBP30-44, MBP83-99, MBP131-145 and MBP140-154 peptides (SEQ ID Nos. 1, 2, 3 and 4).

In a preferred embodiment the invention provides a method for treating demyelination in a subject comprising administering to the subject MBP30-44, MBP83-99, MBP131-145 and MBP140-154 peptides, preferably a composition comprising MBP30-44, MBP83-99, MBP131-145 and MBP140-154 peptides (SEQ ID Nos:1, 2, 3 and 4).

In another aspect said peptide is selected from SEQ ID Nos. 7, 8, 9 and 10. Peptides of SEQ ID Nos. 7, 8, 9 and 10 may be administered to said subject.

In a further aspect the peptide is selected from SEQ ID Nos. 12, 16, 18, 23, 24, 25, 26, 27, 28, 29, 30 and 31.

### DESCRIPTION OF THE FIGURES

FIG. 1 depicts the mechanism by which demyelination occurs (after Oliver Neuhaus, et al, Trends in Pharmacological Sciences Volume 24, Issue 3, Pages 131-138 (March 2003)).

FIG. 2 shows a significant improvement in cognition assessed by PASAT value that underpins a strong trend towards overall disability reduction measured using the MSFC score. Cognition data shown are median+interquartile range; P=0.0101 Wilcoxon matched-pairs signed rank test two-tailed.

FIG. 3 shows that the improvement in cognition is larger in those subjects that start the study with relatively lower scores.

FIG. 4 shows dose-dependent secretion of cytokines in the serum of DR2/Ob1Het/Het mice 2 h after s.c. injection of MBP. Data were analyzed by ANOVA followed by Dunnett's multi-comparison test. * and ** indicate p<0.05 and 0.01, respectively versus the phosphate buffered saline (PBS)-treated group.

FIG. 5 shows time-course of cytokine release in serum after s.c. treatment with ATX-MS-1467 at 100 μg/mouse. Data were analyzed by ANOVA followed by Dunnett's multi-comparison test. *, **, *** and **** indicate p<0.05, 0.01, 0.001 and 0.0001, respectively versus the PBS-treated group.

FIG. 6 shows Serum cytokine levels 2 h after a single or multiple treatments with ATX-MS-1467 at 100 μg/mouse. DR2/Ob1Het/Het mice received from 1 to 10 treatments with ATX-MS-1467 following a 3×/weekly regimen. Data were analyzed by ANOVA followed by Dunnett's multi-comparison test. *, **, *** and **** indicate p<0.05, 0.01, 0.001 and 0.0001, respectively versus the PBS-treated group.

FIG. 7 shows serum cytokine levels 2 h after challenge with PBS or ATX-MS-1467 (100 μg/mouse) following a course of 10 doses of ATX-MS-1467 (100 μg/mouse, 3× weekly) separated from the challenge by a period lasting 2, 7, 14 or 21 days during which the mice did not receive any treatment. The length of the wash-out period is indicated by the arrows. Data were analysed by ANOVA followed by Dunnett's multi-comparison test. *, **, *** and **** indicates p<0.05, 0.01, 0.001 and 0.0001, respectively versus the PBS-treated group. #, ##, ### and #### indicate p<0.05, 0.01, 0.001 and 0.0001, respectively versus the group receiving a single treatment with ATX-MS-1467

FIG. 8 shows serum cytokine levels 2 h after challenge with PBS or MBP (300 μg/mouse) following a course of 10 doses of ATX-MS-1467 (100 μg/mouse, 3× weekly) or HLAbp (25 μg/mouse, 3× weekly) separated from the challenge by a period lasting 2, 7, 21 or 42 days during which the mice did not receive any treatment. The length of the wash-out period is indicated by the arrows. Data were analyzed by ANOVA followed by Dunnett's multi-comparison test. ** and **** indicate p<0.05 and 0.0001, respectively versus the PBS-challenged group. # and ## indicate p<0.05 and 0.01, respectively versus the untolerized group (i.e. challenge with MBP in the absence of previous treatment).

FIG. 9 shows % of Lag3-expressing CD4+ lymphocytes from spleen of DR2/Ob1Het/Het mice immunized with SCH and treated with either PBS or ATX-MS-1467 as described in the methods. *** indicates p<0.001 versus the PBS-treated group.

FIG. 10 shows prophylactic treatment with ATX-MS-1467 delayed disease onset in the Lewis rat EAE model.(A) Daily clinical score measures with ATX-MS-1467 treatment once or three times weekly starting 3 weeks before disease induction (immunization). (B) Incidence of disease (CS>1) with ATX-MS-1467 treatment once or three times weekly starting 3 weeks before disease induction. *p, 0.05 versus vehicle using Kruskal-Wallis with Dunn's post hoc analysis of comparisons. ^{†}indicates significance versus vehicle treatment determined using the log-rank (Mantel-Cox) test. CS, clinical score; EAE, experimental autoimmune encephalomyelitis; qw, once weekly; SEM, standard error of the mean; tiw, three times weekly; veh, vehicle.

FIG. 11 shows ATX-MS-1467 significantly reduced disease severity in SCH-induced EAE in double-transgenic ‘humanized’ mice. (A) Daily clinical score measures with ATX-MS-1467 treatment twice weekly starting on Day 0 (immunization). (B) Twice-weekly treatment with ATX-MS-1467 starting after initial signs of paralysis. *p, 0.05 versus vehicle. **p, 0.01 versus vehicle.biw, twice weekly; EAE, experimental autoimmune encephalomyelitis; SCH, spinal cord homogenate; SEM, standard error of the mean.

FIG. 12 shows ATX-MS-1467 reduced disease severity in double-transgenic humanized mice more effectively than MBP₈₂₋₉₈ or GA treatment. (A,B) Once-weekly dosing with ATX-MS-1467 (100 μg/mouse), MBP₈₂₋₉₈ (12 μg or 100 μg/mouse), or vehicle control from Day 0. (C,D) Treatment with ATX-MS-1467 (100 μg/mouse, twice weekly) starting on Day 0 significantly reduced EAE compared with vehicle or GA (75 μg/mouse, daily). *p, 0.05 versus vehicle or GA. **p, 0.01 versus vehicle. biw, twice weekly; EAE, experimental autoimmune encephalomyelitis; GA, glatiramer acetate; HED, human equivalent dose; MBP, myelin basic protein; qw, once weekly; SEM, standard error of the mean.

FIG. 13 shows ATX-MS-1467 treatment from Day 0 (immunization) reduced EAE-induced immune cell populations in the central nervous system in double-transgenic humanized mice. (A) Clinical score. (B-E) Cellular infiltrates from spinal cords harvested on Day 15. *p, 0.05 versus vehicle or GA. ***p, 0.001 versus vehicle. EAE, experimental autoimmune encephalomyelitis; qw, once weekly; SEM, standard error of the mean.

FIG. 14 shows Dose-dependent attenuation in disease severity of SCH-induced EAE in DR2/Ob1^{het/het} mice after preventative dosing with ATX-MS-1467 from dpi7. A: comparison of active versus control group by Kruskal Wallis followed by Dunn's test. B: comparison of active versus control by Log-rank test. Group sizes: n=10-14. (*=P<0.05, ***=P<0.001).

FIG. 15 shows effect of treatment with ATX-MS-1467 on spinal cord cytokine concentrations in SCH-induced EAE in DR2/Ob1^{het/het} mice. #, ## and ###=P<0.05, 0.01 and 0.001, respectively by ANOVA followed by Bonferroni versus mice treated with vehicle. Group sizes (n=7-13). C-X-C motif chemokine (CXCL), Interleukin (IL), Interferon (IFN), monocyte chemotactic protein-1 (MCP-1).

FIG. 16 shows effect of ATX-MS-1467 in SCH-induced EAE in DR2/Ob1^{Het/Het} mice with preventative (starting at dpi7) or therapeutic (starting at dpi14) dosing paradigms. *, ** and ***=P<0.05, 0.01 and 0.001, respectively by Kruskal Wallis followed by Dunn's test versus control group. Group sizes (n=20−28).

FIG. 17 shows pathological changes in the spinal cord after preventative (starting at dpi7) or therapeutic (starting at dpi14) treatment with ATX-MS-1467. ***=P<0.001 by ANOVA followed by Bonferroni versus mice treated with vehicle (n=18−20). Luxol fast blue (LFB).

FIG. 18 shows characterization of BBB leakage in SCH-EAE in DR2/Ob1^{het/het}. Clinical scores (A) were measured and imaging performed at indicated time points. BBB leakage was detected in 25% of mice at dpi7 and in 100% of mice of subsequent time points (not shown). The total volume of Gd+leakage in the cerebellum increased between dpi10 and 14 (B) whereas the intensity of the signal was comparable at all time points (C). Representative T1-weighted Gd+ change in signal (indicated by red arrows) at dpi 22 (D).

FIG. 19 shows effect of prophylactic ATX-MS-1467 treatment on BBB leakage. Prophylactic treatment from dpi0 with ATX-MS-1467 prevented BBB leakage in SCH EAE in DR2/Ob1het/het mice (A-C). ATX-MS-1467-treated mice displayed reduced disease severity as compared with PBS-treated mice in terms of clinical scores, total volume of cerebellar leakage and intensity of Gd+within lesions. A significant correlation between clinical scores vs leakage volume (r2=0.48, F=0.57) and vs Gd+intensity (r2=0.57, F=17.3) was observed (D). Representative Gd+MRI at dpi14 (E).

FIG. 20 shows antigen-specific cytokine secretion ex-vivo. *, ** and ***=P<0.05, 0.01 and 0.001, respectively by two-way ANOVA followed by Bonferroni's post tests. Group sizes (n=5−6). Interleukin (IL), Interferon (IFN). *, ** and ***=P<0.05, 0.01 and 0.001, respectively by two-way ANOVA followed by Bonferroni's post tests. Group sizes (n=5-6). Interleukin (IL), Interferon (IFN).

### DETAILED DESCRIPTION

### Apitopes

The present inventors have previously determined that there is a link between the capacity of a peptide to bind to an MHC molecule and be presented to a T cell without further processing, and the peptide's capacity to induce tolerance in vivo (WO 02/16410). If a peptide is too long to bind the peptide binding groove of an MHC molecule without further processing (e.g. trimming), or binds in an inappropriate conformation then it will not be tolerogenic in vivo. If, on the other hand, the peptide is of an appropriate size and conformation to bind directly to the MHC peptide binding groove and be presented to a T cell, then this peptide can be predicted to be useful for tolerance induction.

Apitopes (Antigen Processing-Independent epiTOPES) are capable of binding to an MHC molecule and stimulating a response from T cells without further antigen processing.

The present inventors have shown previously that apitopes derivable from MBP, MOG or PLP are capable of inducing tolerance (see for example WO2002/016410, WO2003/064464, WO2009/056833, WO2014/111841 and WO2014/111840 which are herein incorporated by reference).

The present Examples demonstrate that MBP peptides when administered to a subject unexpectedly lead to a significant improvement in impaired cognition in those subjects. As MOG and PLP apitopes have been shown to have similar properties to MBP, it can be expected that the same effects on impaired cognition will be achieved with MOG and PLP apitopes. The present invention is therefore directed to peptides from MBP, MOG and PLP which function as apitopes for uses or methods for treating impaired cognition, dementia and/or demyelination as described herein.

### Myelin

Myelin is a dielectric (electrically insulating) material that forms a layer, the myelin sheath, usually around only the axon of a neuron. It is essential for the proper functioning of the nervous system. Some of the proteins that make up myelin are myelin basic protein (MBP), myelin oligodendrocyte glycoprotein (MOG), and proteolipid protein (PLP).

### Myelin Basic Protein (MBP)

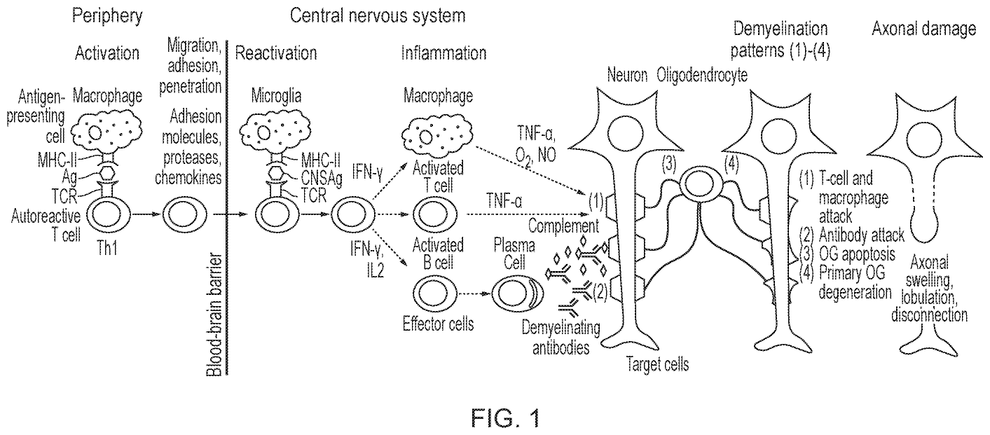

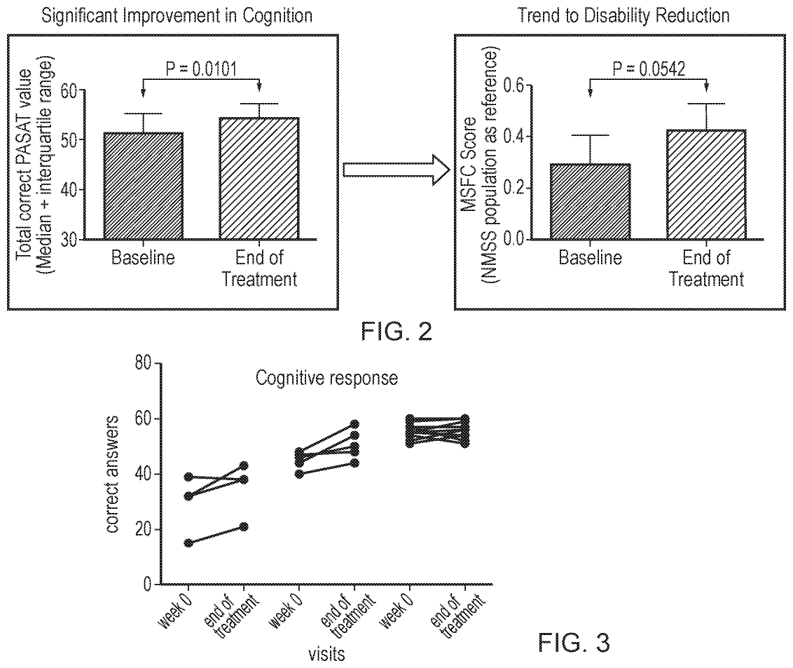

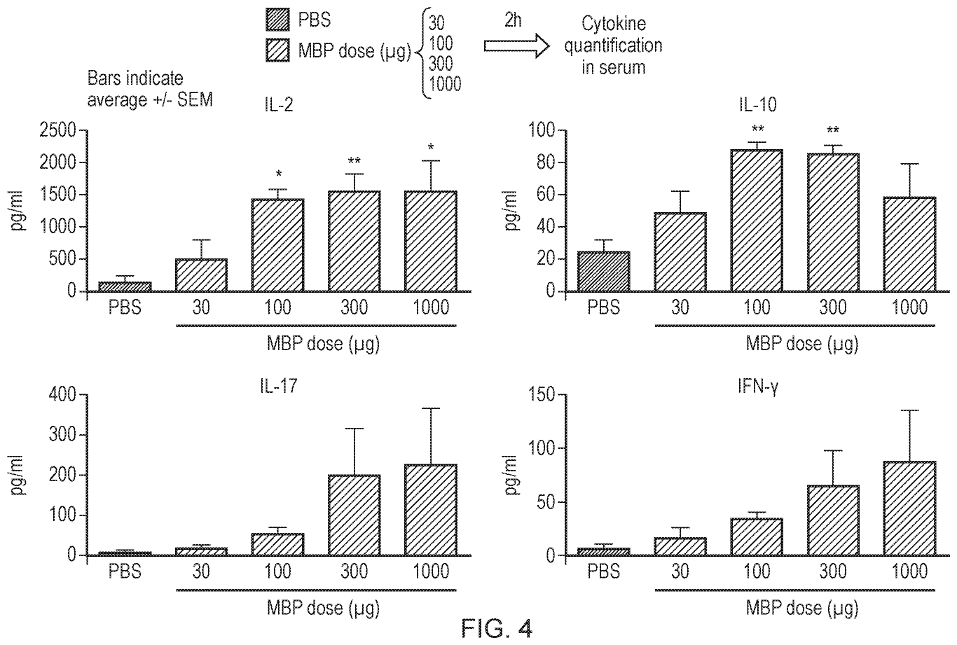

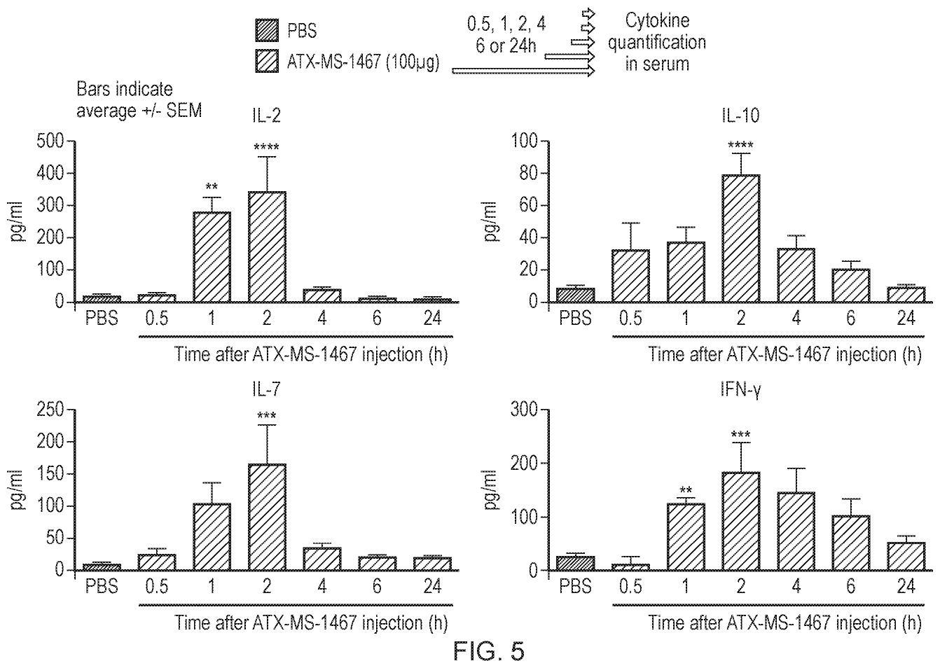

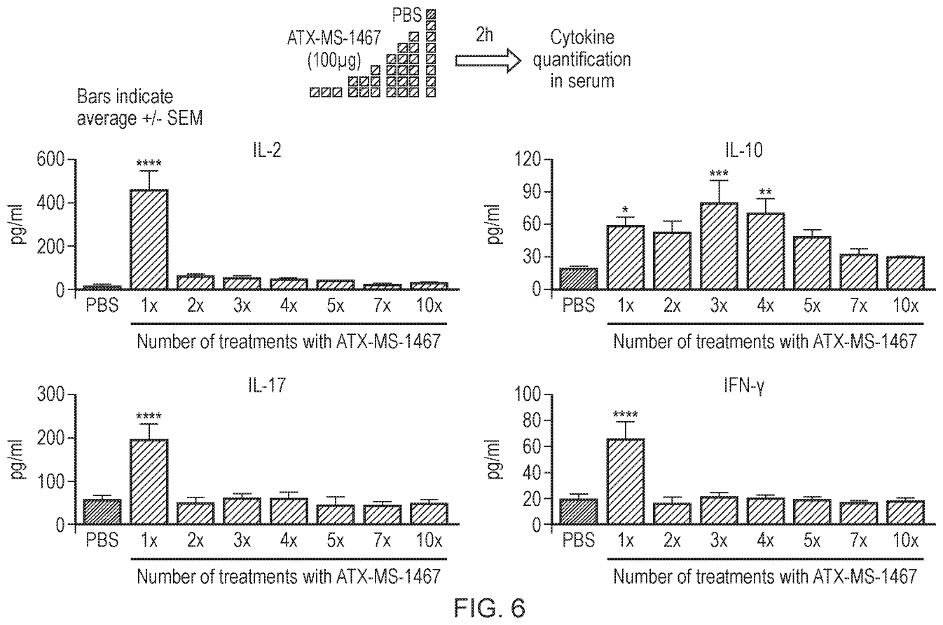

Myelin basic protein (MBP) is an 18.5 kDa protein isolatable from human brain white matter. The mature protein has 170 amino acids and the sequence is widely available in the literature (see for example: Chou et al (1986) J. Neurochem. 46:47-53, FIG. 1; Kamholz et al (1986), PNAS 83:4962-4966, FIG. 2; U.S. Pat. No. 5,817,629, SEQ ID NO: 1; Roth et al (1987), J. Neurosci. Res. 17:321-328, FIG. 4; Medeveczky et al (2006), FEBS Letters 580:545-552, FIG. 3B). Suitable MBP peptides for use according to the present invention are described, for example, in WO2002/016410, WO2003/064464 and WO2009/056833, which are herein incorporated by reference.

Thus, peptides that can be used according to the present invention may be as follows:

MBP 30-44:
(SEQ ID No. 1)
H-Pro-Arg-His-Arg-Asp-Thr-Gly-Ile-Leu-Asp-Ser-Ile-
Gly-Arg-Phe-NH₂
MBP 83-99:
(SEQ ID No. 2)
H-Glu-Asn-Pro-Val-Val-His-Phe-Phe-Lys-Asn-Ile-Val-
Thr-Pro-Arg-Thr-Pro-NH₂
MBP 131-145:
(SEQ ID No. 3)
H-Ala-Ser-Asp-Tyr-Lys-Ser-Ala-His-Lys-Gly-Phe-Lys-
Gly-Val-Asp-NH₂
MBP 140-154:
(SEQ ID No. 4)
H-Gly-Phe-Lys-Gly-Val-Asp-Ala-Gln-Gly-Thr-Leu-Ser-
Lys-Ile-Phe-NH₂

The terms “MBP 30-44”, “MBP 83-99”, “MBP 131-145” and “MBP 140-154” may also encompass modified peptides. For example the peptides may be mutated, by amino acid insertion, deletion or substitution, so long as the MHC binding-specificity of the unmodified peptide is retained, together with its capacity to be presented to a T cell. The peptide may, for example, have 5, 4, 3, 2, 1 or 0 mutations from the unmodified sequence.

Alternatively (or in addition) modifications may be made without changing the amino acid sequence of the peptide. For example, D-amino acids or other unnatural amino acids can be included, the normal amide bond can be replaced by ester or alkyl backbone bonds, N- or C-alkyl substituents, side chain modifications, and constraints such as disulphide bridges and side chain amide or ester linkages can be included. Such changes may result in greater in vivo stability of the peptide, and a longer biological lifetime.

Modification of epitopes may be performed based on predictions for more efficient T-cell induction derived using the program “Peptide Binding Predictions” devised by K. Parker (NIH) which may be found at http://www-bimas.dcrt.nih.gov/cgi-bin/molbio/ken_parker_comboform (see also Parker, K. C et al. 1994. J. Immunol. 152:163).

MBP peptides as described herein may be formulated into a composition as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

In the methods and uses of the invention described herein the peptide or composition may be administered following a dose-escalation protocol. In a “dose escalation” protocol a plurality of doses is given to the patient in ascending concentrations. Such an approach has been used, for example, for phospholipase A2 peptides in immunotherapeutic applications against bee venom allergy (Müller et al (1998) J. Allergy Clin Immunol. 101:747-754 and Akdis et al (1998) J. Clin. Invest. 102:98-106).

In one aspect, the peptide may be administered in a dose escalation protocol in the following doses:

Day 1: a first dose of about 15 to about 40 μg;

Day 14±7 days: a second dose of about 35-65 μg;

Day 28±7 days: a third dose of about 80-120 μg;

Day 42±7 days: a fourth dose of about 300-500 μg;

Day 56±7 days: a fifth dose of about 400-2000 μg;

Day 70±7 days: a sixth dose of about 400-2000 μg;

Day 84±7 days: a seventh dose of about 400-2000 μg;

Day 98±7 days: an eighth dose of about 400-2000 μg;

Day 112±7 days: a ninth dose of about 400-2000 μg; and

Day 126±7 days: a tenth dose of about 400-2000 μg.

In one aspect the peptides may be administered as follows:

Day 1: a first dose of about 25 μg;

Day 14: a second dose of about 50 μg;

Day 28: a third dose of about 100 μg;

Day 42: a fourth dose of about 400 μg;

Day 56: a fifth dose of about 800 μg;

Day 70: a sixth dose of about 800 μg;

Day 84: a seventh dose of about 800 μg;

Day 98: a eighth dose of about 800 μg;

Day 112: a ninth dose of about 800 μg; and

Day 126: a tenth dose of about 800 μg.

In an alternative aspect, a first dose of about 50 μg may be administered on day 1, followed by a second dose of about 200 μg on day 15, followed by a third dose of about 800 μg on day 29. Subjects may also in one aspect receive a dose of about 800 μg approximately every two weeks, or every 14 days, thereafter, for example for a period of at least 16 weeks.

It has been found that two of the peptides, MBP 30-44 and 131-145, are HLA-DQ6 binding and two are HLA-DR2 binding (MBP 140-154 and 83-99). The combined use of these apitopes provides more widespread cover of the different Major Histocompatibility Complex (MHC) haplotypes seen in MS patients than therapy with a single peptide.

### Myelin Oligodendrocyte Glycoprotein (MOG)

Myelin oligodendrocyte glycoprotein (MOG) is a type I integral membrane protein possessing a single extracellular Ig variable domain (Ig-V). The amino acid sequence of MOG is highly conserved among animal species (>90%), indicative of an important biological function. MOG is specifically expressed in the CNS on the outermost lamellae of the myelin sheath as well as the cell body and processes of oligodendrocytes.

The sequence of mature MOG (lacking the 29 amino acid signal peptide) is given below (SEQ ID No. 5).

SEQ ID No. 5
GQFRVIGPRHPIRALVGDEVELPCRISPGKNATGMEVGWYRPPFSRVVHLY
RNGKDQDGDQAPEYRGRTELLKDAIGEGKVTLRIRNVRFSDEGGFTCFFRD
HSYQEEAAMELKVEDPFYWVSPGVLVLLAVLPVLLLQITVGLVFLCLQYRL
RGKLRAEIENLHRTFDPHFLRVPCWKITLFVIVPVLGP LVALIICYNWLH
RRLAGQFLEELRNPF

Peptides for use according to the present invention may be derivable from region 40-60 of myelin oligodendrocyte glycoprotein. Peptides may be derivable from a fragment of the antigen which arises by natural processing of the antigen by an antigen presenting cell.

Region 40-60 of MOG has the following sequence:

SEQ ID No. 6
YRPPFSRVVHLYRNGKDQDGD

The peptide may comprise the minimal epitope from the following peptides: MOG 41-55, 43-57, 44-58 and 45-59.

The sequences of MOG 41-55, 43-57, 44-58 and 45-59 are:

MOG 41-55:
(SEQ ID No. 7)
RPPFSRVVHLYRNGK
MOG 43-57:
(SEQ ID No. 8)
PFSRVVHLYRNGKDQ
MOG 44-58:
(SEQ ID No. 9)
FSRVVHLYRNGKDQD
MOG 45-59:
(SEQ ID No. 10)
SRVVHLYRNGKDQDG

Peptides comprising SEQ ID Nos. 7, 8, 9 and/or 10 may be used according to the methods and uses of the invention as described herein. In one aspect the peptide or peptides consist of SEQ ID Nos. 7, 8, 9 and/or 10.

The peptide for use according to the present invention may comprise the minimal epitope from MOG 41-55. The peptide may consist of MOG 41-55 (SEQ ID No. 7).

### Myelin Proteolipid Protein (PLP)

Myelin proteolipid protein (PLP), the most abundant protein of central nervous system (CNS) myelin, is a hydrophobic integral membrane protein.

The sequence of human PLP is shown in SEQ ID No. 11:

| | |
|---|---|
| 1 | GLLECCARC LVGAPFASLV ATGLCFFGVA LFCGCGHEAL TGTEKLIETY FSKNYQDYEY |
| 60 | LINVIHAFQY VIYGTASFFF LYGALLLAEG FYTTGAVRQI FGDYKTTICG KGLSATVTGG |
| 120 | QKGRGSRGQH QAHSLERVCH CLGKWLGHPD KFVGITYALT VVWLLVFACS AVPVYIYFNT |
| 180 | WTTCQSIAFP SKTSASIGSL CADARMYGVL PWNAFPGKVC GSNLLSICKT AEFQMTFHLF |
| 240 | IAAFVGAAAT LVSLLTFMIA ATYNFAVLKL MGRGTKF |

The peptides for use according to the present invention may be derivable from a hydrophilic region of the PLP sequence. The peptide may be derivable from a fragment of the antigen which arises by natural processing of the antigen by an antigen presenting cell.

Peptides derivable from the hydrophilic regions of PLP are:

PLP 36-61:
(SEQ ID No. 12)
HEALTGTEKLIETYFSKNYQDYEYLI
PLP 88-119:
(SEQ ID No. 13)
EGFYTTGAVRQIFGDYKTTICGKGLSATVTGG
PLP 104-135:
(SEQ ID No. 14)
KTTICGKGLSATVTGGQKGRGSRGQHQAHSLE
PLP 119-150:
(SEQ ID No. 15)
GQKGRGSRGQHQAHSLERVCHCLGKWLGHPDK
PLP 179-206:
(SEQ ID No. 16)
TWTTCQSIAFPSKTSASIGSLCADARMY
PLP 192-219:
(SEQ ID No. 17)
TSASIGSLCADARMYGVLPWNAFPGKVC
PLP 207-234:
(SEQ ID No. 18)
GVLPWNAFPGKVCGSNLLSICKTAEFQM
PLP 260-276:
(SEQ ID No. 19)
ATYNFAVLKLMGRGTKF

The peptide may comprise all or a portion of the following proteolipid protein (PLP) regions:

PLP 36-61:
(SEQ ID No. 12)
HEALTGTEKLIETYFSKNYQDYEYLI
PLP 179-206:
(SEQ ID No. 16)
TWTTCQSIAFPSKTSASIGSLCADARMY
PLP 192-219:
(SEQ ID No. 17)
TSASIGSLCADARMYGVLPWNAFPGKVC
PLP 207-234:
(SEQ ID No. 18)
GVLPWNAFPGKVCGSNLLSICKTAEFQM.

The peptide may comprise a minimal epitope from one of these regions.

The peptide may comprise a portion of the following regions:

PLP 39-57:
(SEQ ID No. 20)
LTGTEKLIETYFSKNYQDY
PLP 180-198:
(SEQ ID No. 21)
WTTCQSIAFPSKTSASIGS
PLP 208-222:
(SEQ ID No. 22)
VLPWNAFPGKVCGSN

In one aspect the peptide may be selected from the following PLP peptides:

PLP 39-53:
(SEQ ID No. 23)
LTGTEKLIETYFSKN
PLP 42-56:
(SEQ ID No. 24)
TEKLIETYFSKNYQD
PLP 43-57:
(SEQ ID No. 25)
EKLIETYFSKNYQDY
PLP 180-194:
(SEQ ID No. 26)
WTTCQSIAFPSKTSA
PLP 181-195:
(SEQ ID No. 27)
TTCQSIAFPSKTSAS
PLP 182-196:
(SEQ ID No. 28)
TCQSIAFPSKTSASI
PLP183-197:
(SEQ ID No. 29)
CQSIAFPSKTSASIG
PLP 184-198:
(SEQ ID No. 30)
QSIAFPSKTSASIGS
PLP 208-222:
(SEQ ID No. 31)
VLPWNAFPGKVCGSN
PLP 36-61:
(SEQ ID No. 12)
HEALTGTEKLIETYFSKNYQDYEYLI
PLP 179-206:
(SEQ ID No. 16)
TWTTCQSIAFPSKTSASIGSLCADARMY
and
PLP 207-234:
(SEQ ID No. 18)
GVLPWNAFPGKVCGSNLLSICKTAEFQM.

The peptide may comprise a minimal epitope from one of these peptides.

In particular the peptide may comprise, consist of, or comprise the minimal epitope, from one of the following:

PLP 39-53:
(SEQ ID No. 23)
LTGTEKLIETYFSKN
PLP 181-195:
(SEQ ID No. 27)
TTCQSIAFPSKTSAS
PLP 179-206:
(SEQ ID No. 16)
TWTTCQSIAFPSKTSASIGSLCADARMY.

### Demyelinating Diseases

The disease to be treated according to the present invention is a demyelinating disease. Such diseases may include Alzheimer's disease and Parkinson's disease, as discussed above. Such diseases may also include vanishing white matter disease and multiple sclerosis (MS). In one aspect the disease is a multiple sclerosis.

### Multiple Sclerosis

Multiple sclerosis (MS) is a chronic degenerative disease of the central nervous system (CNS) in which the immune system, normally charged with fighting off invading organisms, attacks the body's myelin sheaths, the protective insulation that envelops neurons and facilitates high-speed neuronal communication. Without myelin to assist and protect neurons, the brain and spinal cord signals that permit us to interact with our environment malfunction.

MS may cause numerous physical and mental symptoms, and often progresses to both physical and cognitive disability. Disease onset usually occurs in young adults (20-40 yrs), is more common in women, and affects more than 1 million people around the world. MS is currently believed to be an immune-mediated disorder in which the body's own immune system attacks and damages myelin.

The disease course of MS is varied and may lie dormant or progress steadily over time. Several subtypes of MS have been described based on patterns of progression.

Depending on the extent and location of damage in the CNS, patients with MS may experience a wide variety of symptoms. The most commonly reported symptoms at the time of diagnosis are blurred vision, tingling and/or numbness, and loss of coordination. As the disease progresses, patients with MS commonly experience fatigue, spasticity, difficulty walking, and cognitive impairment. Before 1993 there were no approved treatments of MS. Today, eight of the nine FDA-approved disease-modifying treatments are designed to reduce the frequency of clinical exacerbations in MS, and one is approved to improve walking ability. None, however, target the cognitive impairment often seen in people who have MS.

There are four subtypes of MS, defined by disease progression. Relapsing-remitting MS (RR-MS) is the most common; this subtype is the initial diagnosis of approximately 85 percent of all people with MS. In RR-MS, patients experience flare-ups of disease symptoms for a period of time, followed by a complete recovery or remission. The majority of patients diagnosed with RR-MS develop secondary-progressive MS (SP-MS) within 10 to 20 years. In SP-MS, as in RR-MS, patients experience flare-ups or relapses of disease symptoms, but there is a steady increase in disease severity between the relapses. The second most common subtype diagnosed at initial presentation is primary-progressive MS (PP-MS), in which a patient experiences a steady increase in symptom severity from the time of disease onset. The final and most rare subtype of MS, progressive-relapsing MS (PR-MS), involves intermittent relapses punctuating a steady progression of the disease. While patients with progressive subtypes of MS are more likely to experience cognitive impairment in general, further studies of patients with PP-MS and PR-MS are needed. Earlier onset of MS increases a patient's chance of developing MS-related cognitive decline.

The subject to be treated according to the present invention may have MS. In one aspect the subject has relapsing-remitting MS.

In one aspect the subject may have secondary-progressive MS. In one aspect the subject may have primary-progressive MS. In one aspect the subject may have progressive-relapsing MS.

### Impaired Cognition

Disability in MS may be measured by the Multiple Sclerosis Functional Composite (MSFC) score. MSFC is a method known in the art for quantifying neurological function, and is described in Cutter et al. Brain (1999) 122, 871-882, which is herein incorporated by reference. A manual on MSFC administration and scoring is also available from the National Multiple Sclerosis Society (revised October 2001) and prepared by Fisher J., Jak A., Kniker J., Rudick R. and Cutter G. This manual is also incorporated herein by reference.

One of the aspects quantified by MSFC is cognition. In the clinical trial carried out by the present inventors disability was firstly measured using the MSFC score. It was found that disability was significantly reduced in the treatment group compared with baseline, and that the disability improvement was largely due to a significant improvement in impaired cognition.

The concept of cognitive impairment is known in the art, for example as discussed in Rahn et al. Cerebrum 2012:14.

“Cognition” refers to a range of high-level brain functions including the ability to learn and remember information, organize, plan and problem-solve, focus, maintain and shift attention, understand and use language, accurately perceive the environment, and perform calculations.

Cognitive changes are a common symptom of MS—more than half of all people with MS will develop problems with cognition. For some, it may even be the first symptom of MS. Certain functions are more likely to be affected than others:
Information processing (dealing with information gathered by the five senses)
Memory (acquiring, retaining and retrieving new information)
Attention and concentration (particularly divided attention)
Executive functions (planning and prioritizing)
Visuospatial functions (visual perception and constructional abilities)
Verbal fluency (word-finding)

Cognitive impairment may occur at all stages of MS. Subjects with cognitive problems may notice one or more of the following symptoms:
Problems remembering events or conversations
Problems remembering names
Problems with multitasking
Problems with learning new materials
Problems with attention span
Problems with learning directions
Problems with decision-making

Cognitive impairment may be assessed using the “PASAT” test (Paced Auditory Serial Addition Test). This test is known in the art, and is a component of the MSFC. The test and how to administer it will be known to one skilled in the art in this field. Essentially, the PASAT is a measure of cognitive function that assesses auditory information processing speed and flexibility, as well as calculation ability. It was developed by Gronwell in 1977 and later adapted by Rao and colleagues in 1989 for use in MS. The PASAT test is presented using audio cassette tape or compact disk to ensure standardization in the rate of stimulus presentation. Single digits are presented every 3 seconds and the patient must add each new digit to the one immediately prior to it. Shorter inter-stimulus intervals, e.g., 2 seconds or less have also been used with the PASAT but tend to increase the difficulty of the task. Two alternate forms have been developed to minimize possible familiarity with the stimulus items when the PASAT is repeated over more than one occasion.

The methods according to the present invention may be used before or after or in combination with other treatments for demyelinating diseases such as multiple sclerosis.

In a preferred embodiment of the present invention, the subject of any of the methods of the invention is a mammal, preferably a cat, dog, horse, donkey, sheep, pig, goat, cow, mouse, rat, rabbit or guinea pig, but most preferably the subject is a human.

As defined herein “treatment” refers to reducing, alleviating or eliminating one or more symptoms of impaired cognition, relative to the symptoms prior to treatment.

“Prevention” (or prophylaxis) refers to delaying or preventing the onset of cognitive impairment. For example, prevention may refer to early intervention before there is evidence of cognitive decline, but a risk of neuronal loss leading to cognitive decline. Prevention may be absolute or may be effective only in some individuals or for a limited amount of time.

By “treating impaired cognition” as used herein is intended to mean an improvement in any aspect of impaired cognition, such as nut not limited to any of the aspects of cognition described herein. By way of non-limiting example these may include information processing, memory, attention and concentration, executive functions, visuospatial functions and/or verbal fluency.

### Dementia

The word ‘dementia’ describes a set of symptoms that may include memory loss and difficulties with thinking, problem-solving or language. These changes are often small to start with, but for someone with dementia they have become severe enough to affect daily life. A person with dementia may also experience changes in their mood or behaviour.

Dementia may be caused when the brain is damaged by diseases, such as Alzheimer's disease or a series of strokes. Alzheimer's disease is a common cause of dementia, but not the only one. The specific symptoms that someone with dementia experiences will depend on the parts of the brain that are damaged and the disease that is causing the dementia.

The number of people with dementia is steadily increasing.

It is estimated that there are 850,000 people with dementia in the UK, with numbers set to rise to over 1 million by 2025. This is projected to increase to 2 million by 2051. 225,000 will develop dementia this year, equating to approximately one every three minutes. 1 in 6 people over the age of 80 have dementia.

Each person is unique and will experience dementia in their own way. Different types of dementia also tend to affect people differently, especially in the early stages.

A person with dementia may have cognitive symptoms (to do with thinking or memory), and may have problems with some of the following:
day-to-day memory—for example, difficulty recalling events that happened recently
concentrating, planning or organising—for example, difficulties making decisions, solving problems or carrying out a sequence of tasks (such as cooking a meal)
language—for example, difficulties following a conversation or finding the right word for something
visuospatial skills—for example, problems judging distances (such as on stairs) and seeing objects in three dimensions
orientation—for example, losing track of the day or date, or becoming confused about where they are.

A person with dementia may also have changes in their mood. For example, they may become frustrated or irritable, apathetic or withdrawn, anxious, easily upset or unusually sad. With some types of dementia, the person may see things that are not really there (visual hallucinations) or strongly believe things that are not true (delusions).

Dementia is progressive, i.e. the symptoms gradually get worse over time. How quickly this happens varies greatly from person to person. As dementia progresses, the person may develop behaviours that seem unusual or out of character. These behaviours may include asking the same question over and over, pacing, restlessness or agitation.

A person with dementia, especially in the later stages, may have physical symptoms such as muscle weakness or weight loss. Changes in sleep pattern and appetite are also common.

As defined herein “treatment” refers to reducing, alleviating or eliminating one or more symptoms of dementia, relative to the symptoms prior to treatment. Such symptoms include, but are not limited to, any of the symptoms described herein.

“Prevention” (or prophylaxis) refers to delaying or preventing the onset of dementia. For example, prevention may refer to early intervention before there is evidence of dementia, but a risk of neuronal loss leading to dementia. Prevention may be absolute or may be effective only in some individuals or for a limited amount of time.

By “treating dementia” as used herein is intended to mean an improvement in any aspect of dementia, such as but not limited to any of the aspects described herein.

There is no single test for dementia. Diagnosis may be based on a combination of things:
taking a ‘history’—the doctor talking to the person and someone who knows them well about how their problems developed and how they are now affecting their daily life
physical examination and tests (for example, blood tests) to exclude other possible causes of the person's symptoms
tests of mental abilities (for example, memory, thinking)—simpler tests will be carried out by a nurse or doctor, more specialist tests by a psychologist
a scan of the brain, if this is needed to make the diagnosis.

There are different forms of dementia that have common underlying pathology/causes:

Alzheimer's disease—This is the most common cause of dementia. Problems with day-to-day memory are often the first thing to be noticed, but other symptoms may include difficulties finding the right words, solving problems, making decisions, or perceiving things in three dimensions.

Vascular dementia—If the oxygen supply to the brain is reduced because of narrowing or blockage of blood vessels, some brain cells become damaged or die. This is what happens in vascular dementia. The symptoms can occur suddenly, following one large stroke. Or they can develop over time, because of a series of small strokes. Vascular dementia can also be caused by disease affecting the small blood vessels deep in the brain, known as subcortical vascular dementia. The symptoms of vascular dementia vary and may overlap with those of Alzheimer's disease. Many people have difficulties with problem-solving or planning, thinking quickly and concentrating. They may also have short periods when they get very confused.

Mixed dementia—This is when someone has more than one type of dementia, and a mixture of the symptoms of those types. It is common for someone to have both Alzheimer's disease and vascular dementia together.

Dementia with Lewy bodies—This type of dementia involves tiny abnormal structures (Lewy bodies) forming inside brain cells. They disrupt the chemistry of the brain and lead to the death of brain cells. Early symptoms can include alertness that varies over the course of the day, hallucinations, and difficulties judging distances. A person's day-to-day memory is usually affected less than in the early stages of Alzheimer's disease. Dementia with Lewy bodies is closely related to Parkinson's disease and often has some of the same symptoms, including difficulty with movement.

Frontotemporal dementia (including Pick's disease)—In frontotemporal dementia, the front and side parts of the brain are damaged. Clumps of abnormal proteins form inside brain cells, causing them to die. At first, changes in personality and behaviour may be the most obvious signs. Depending on which areas of the brain are damaged, the person may have difficulties with fluent speech or forget the meaning of words.

There are many other diseases that can lead to dementia. These are rare—together they account for only about 5 percent of all dementia. They tend to be more common among younger people with dementia (under the age of 65). These rarer causes include corticobasal degeneration, progressive supranuclear palsy, HIV infection, Niemann-Pick disease type C, and Creutzfeldt-Jakob disease (CJD).

People with Parkinson's disease or Huntington's disease may also develop dementia as the illness gets worse. People with Down's syndrome are also at a particular risk of developing Alzheimer's disease as they get older.

In one aspect of the invention the subject has Alzheimer's disease or Parkinson's disease.

The methods according to the present invention may be used before, or after, or in combination with other treatments for dementia.

For example, patients with mild to moderate Alzheimer's disease or mixed dementia in which Alzheimer's is the main cause may be prescribed one of three different drugs: donepezil, rivastigmine or galantamine. In the moderate or severe stages of Alzheimer's disease someone may be offered memantine.

Donepezil, rivastigmine and galantamine can also be helpful for someone with dementia with Lewy bodies who has distressing hallucinations or delusions, or who has behaviours that challenge (for example, agitation or aggression).

As regards vascular dementia, drugs may be offered to treat the underlying medical conditions that cause dementia. These conditions often include high blood pressure, high cholesterol, diabetes or heart problems. Controlling these may help slow the progression of dementia.

A wide range of other drugs may be prescribed at different times for a person with dementia. These include drugs for depression or anxiety, sleeping tablets or antipsychotics.

A range of non-drug treatments are also available that can help someone to live well with dementia, including for example information, advice, support, therapies and activities.

The method and uses according to the present invention as described herein may be used in conjunction with existing treatments or therapies for dementia.

In one aspect of the invention the dementia is a result of Alzheimer's disease; that is, the subject has Alzheimer's disease.

In one aspect of the invention the dementia is a result of Parkinson's disease; that is, the subject has Parkinson's disease.

In a preferred embodiment of the present invention, the subject of any of the methods of the invention is a mammal, preferably a cat, dog, horse, donkey, sheep, pig, goat, cow, mouse, rat, rabbit or guinea pig, but most preferably the subject is a human.

Animal models of dementia, Alzheimer's disease or Parkinson's disease treated with the peptides of the invention are expected to exhibit improved clinical scores.

### Demyelination

The present invention can improve, or treat, or prevent demyelination in a subject.

Demyelination results in diverse symptoms determined by the functions of the affected neurons. It disrupts signals between the brain and other parts of the body; symptoms differ from patient to patient, and have different presentations upon clinical observation and in laboratory studies. Typical symptoms include:
blurriness in the central visual field that affects only one eye, may be accompanied by pain upon eye movement
double vision
loss of vision/hearing
odd sensation in legs, arms, chest, or face, such as tingling or numbness (neuropathy)
weakness of arms or legs
cognitive disruption, including speech impairment and memory loss
heat sensitivity (symptoms worsen or reappear upon exposure to heat, such as a hot shower)
loss of dexterity
difficulty coordinating movement or balance disorder
difficulty controlling bowel movements or urination
fatigue
tinnitus

As defined herein “treatment” refers to reducing, alleviating or eliminating one or more symptoms of demyelination, relative to the symptoms prior to treatment. Such symptoms include, but are not limited to, any of the symptoms described herein.

“Prevention” (or prophylaxis) refers to delaying or preventing the onset of demyelination. Prevention may be absolute or may be effective only in some individuals or for a limited amount of time.

By “treating demyelination” as used herein is intended to mean an improvement in demyelination, for example via remyelination of a neuron.

The methods according to the present invention may be used before, or after, or in combination with other treatments for demyelination.

The method and uses according to the present invention as described herein may be used in conjunction with existing treatments or therapies for demyelination.

Demyelination is involved in neurodegenerative autoimmune diseases, such as multiple sclerosis, acute disseminated encephalomyelitis, neuromyelitis optica, transverse myelitis, chronic inflammatory demyelinating polyneuropathy, Guillain-Barré syndrome, central pontine myelinosis, inherited demyelinating diseases such as leukodystrophy, Charcot-Marie-Tooth disease, pernicious anaemia and Canavan disease.

Demyelination may also be involved in dementia, and conditions such as Alzheimer's disease and Parkinson's disease.

In one aspect of the invention the subject has multiple sclerosis.

In one aspect of the invention the subject has Alzheimer's disease.

In one aspect of the invention the subject has Parkinson's disease.

In a preferred embodiment of the present invention, the subject of any of the methods of the invention is a mammal, preferably a cat, dog, horse, donkey, sheep, pig, goat, cow, mouse, rat, rabbit or guinea pig, but most preferably the subject is a human.

Treatment of animal models of demyelinating diseases with the peptides of the invention is expected to facilitate remyelination of neurons.

### Peptide

The term “peptide” is used in the normal sense to mean a series of residues, typically L-amino acids, connected one to the other, typically by peptide bonds between the a-amino and carboxyl groups of adjacent amino acids. The term includes modified peptides and synthetic peptide analogues.

The peptides may be made using chemical methods (Peptide Chemistry, A practical Textbook. Mikos Bodansky, Springer-Verlag, Berlin). For example, peptides can be synthesized by solid phase techniques (Roberge J Y et al (1995) Science 269: 202-204), cleaved from the resin, and purified by preparative high performance liquid chromatography (e.g., Creighton (1983) Proteins Structures And Molecular Principles, WH Freeman and Co, New York, N.Y.). Automated synthesis may be achieved, for example, using the ABI 43 1 A Peptide Synthesizer (Perkin Elmer) in accordance with the instructions provided by the manufacturer.

The peptide may alternatively be made by recombinant means, or by cleavage from a longer polypeptide. For example, the peptide may be obtained by cleavage from the relevant protein, which may be followed by modification of one or both ends. The composition of a peptide may be confirmed by amino acid analysis or sequencing (e.g., the Edman degradation procedure).

In one aspect the peptide for use according to the present invention may have at least about 60, 65, 70, 75, 80, 85, 90, 92, 92, 93, 94, 95, 96, 97, 98, 99 or 100% identity to any of the peptides described herein.

Sequence identity may be assessed by any convenient method. However, for determining the degree of sequence identity between sequences, computer programs that make multiple alignments of sequences are useful, for instance Clustal W (Thompson et al., (1994) Nucleic Acids Res., 22: 4673-4680). Programs that compare and align pairs of sequences, like ALIGN (Myers et al., (1988) CABIOS, 4: 1-17), FASTA (Pearson et al., (1988) PNAS, 85:2444-2448; Pearson (1990), Methods Enzymol., 183: 63-98) and gapped BLAST (Altschul et al., (1997) Nucleic Acids Res., 25: 3389-3402) are also useful for this purpose. Furthermore, the Dali server at the European Bioinformatics institute offers structure-based alignments of protein sequences (Holm (1993) J. Mol. Biol., 233: 123-38; Holm (1995) Trends Biochem. Sci., 20: 478-480; Holm (1998) Nucleic Acid Res., 26: 316-9).

Multiple sequence alignments and percent identity calculations may be determined using the standard BLAST parameters, (using sequences from all organisms available, matrix Blosum 62, gap costs: existence 11, extension 1).

Alternatively, the following program and parameters may be used: Program: Align Plus 4, version 4.10 (Sci Ed Central Clone Manager Professional Suite). DNA comparison: Global comparison, Standard Linear Scoring matrix, Mismatch penalty=2, Open gap penalty=4, Extend gap penalty=1. Amino acid comparison: Global comparison, BLOSUM 62 Scoring matrix.

Also included in the scope of the invention are variants of the stated or given sequences, as long as the variant retains the functional activity of the parent i.e. the variants are functionally equivalent, in other words they have or exhibit an activity of the parent peptide as defined herein. Such variants may comprise amino acid substitutions, additions or deletions (including truncations at one or both ends) of the parent sequence.

Also included are functionally-equivalent derivatives in which one or more of the amino acids are chemically derivitised, e.g. substituted with a chemical group. By a “variant” of the given amino acid sequence is intended to mean that the side chains of, for example, one or two of the amino acid residues may be altered (for example by replacing them with the side chain of another naturally occurring amino acid residue or some other side chain) such that the peptide retains the functional activity of the parent peptide from which it is derived.

Variants may involve the replacement of an amino acid residue by one or more of those selected from the residues of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

Such variants may arise from homologous substitution i.e. like-for-like/conservative substitution such as basic for basic, acidic for acidic, polar for polar etc. Non-homologous substitution may also occur i.e. from one class of residue to another or alternatively involving the inclusion of unnatural amino acids such as ornithine, diaminobutyric acid, norleucine, pyridylalanine, thienylalanine, naphthylalanine and phenylglycine.

A substitution may be a conservative substitution. As used herein, a “conservative substitution” refers to changing amino acid identity at a given position to replace with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S. Also included are functionally-equivalent derivatives in which one or more of the amino acids are chemically derivatised, e.g. substituted with a chemical group. Functionally-equivalent derivatives may be modified chemically by reacting specific amino acids either before or after synthesis of the peptide. Examples are known in the art e.g. as described in R. Lundblad, Chemical Reagents for Protein Modification, 3rd ed. CRC Press, 2004 (Lundblad, 2004. Chemical modification of amino acids includes but is not limited to, modification by acylation, amidination, pyridoxylation of lysine, reductive alkylation, trinitrobenzylation of amino groups with 2,4,6-trinitrobenzene sulphonic acid (TNBS), amide modification of carboxyl groups and sulphydryl modification by performic acid oxidation of cysteine to cysteic acid, formation of mercurial derivatives, formation of mixed disulphides with other thiol compounds, reaction with maleimide, carboxymethylation with iodoacetic acid or iodoacetamide and carbamoylation with cyanate at alkaline pH, although without limitation thereto.

The peptide of the present invention may comprise between 8 and 30 amino acids, for example 8 to 25 amino acids, 8 to 20 amino acids, 8 to 15 amino acids or 8 to 12 amino acids. In one aspect the peptide of the present invention may thus be 8, 9, 10, 11, 12, 13, 14, 15, 16, 17, 18, 19, 20, 21, 22, 23, 24, 25, 26, 27, 28, 29 or 30 amino acids in length.

For practical purposes, there are various other characteristics which the peptide may show. For example, it is important that the peptide is sufficiently stable in vivo to be therapeutically useful. The half-life of the peptide in vivo may be at least 10 minutes, 30 minutes, 4 hours, or 24 hours.

The peptide may also demonstrate good bioavailability in vivo. The peptide may maintain a conformation in vivo which enables it to bind to an MHC molecule at the cell surface without due hindrance.

In one embodiment the peptide may comprise any amino acid that may improve or optimise the druggability of the peptide, for example natural or artificial amino acids can improve solubility of peptides. Suitable modifications will be known to one skilled in the art. We refer, for example, to WO 2015/019302 and WO2014/072958.

For example, the peptide may have the following formula:
KKG/KKK-myelin-derived peptide-GKK/KKK

The peptides may be in the form of a composition, preferably a pharmaceutical composition.

Peptides may be formulated into the composition as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic acid. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

Alternatively (or in addition) if the pharmaceutical composition (or any part thereof) is to be administered in multiple doses, each dose may be packaged separately.

Also, in the pharmaceutical compositions of the present invention, the, or each, peptide may be admixed with any suitable binder(s), lubricant(s), suspending agent(s), coating agent(s), or solubilising agent(s).

### Formulation

The composition according to the invention as described herein may by prepared as an injectable, either as liquid solution or suspension; solid form suitable for solution in, or suspension in, liquid prior to injection may also be prepared. The preparation may also be emulsified, or the peptides encapsulated in liposomes. The peptide may alternatively be encapsulated in a carrier or bound to the surface of a carrier, for example a nanoparticle. The active ingredients may be mixed with excipients which are pharmaceutically acceptable and compatible with the active ingredient. Suitable excipients are, for example, water, saline (for example, phosphate-buffered saline), dextrose, glycerol, ethanol, or the like and combinations thereof.

In addition, if desired, the composition may contain minor amounts of auxiliary substances such as wetting or emulsifying agents and/or pH buffering agents. Buffering salts include phosphate, citrate, acetate. Hydrochloric acid and/or sodium hydroxide may be used for pH adjustment. For stabilisation, disaccharides may be used such as sucrose or trehalose.

In the composition, the relative ratio of the peptides may be approximately 1:1. Alternatively the relative ratios of each peptide may be altered, for example, if it is found that one peptide works better than the others in particular HLA types.

After formulation, the peptides or composition may be incorporated into a sterile container which is then sealed and stored at a low temperature, for example 4° C., or it may be freeze-dried.

Conveniently the composition is prepared as a lyophilised (freeze-dried) powder. Lyophilisation permits long-term storage in a stabilised form. Lyophilisation procedures are well known in the art, see for example http://www.devicelink.com/ivdt/archive/97/01/006.html. Bulking agents are commonly used prior to freeze-drying, such as mannitol, dextran or glycine.

The peptides or composition may be administered in a convenient manner such as by the oral, intravenous (where water soluble), intramuscular, subcutaneous, sublingual, intranasal, intradermal or suppository routes or implanting (e.g. using slow release molecules).

The peptides or composition may advantageously be administered via intranasal, subcutaneous or intradermal routes. In a preferred embodiment administration is intradermal.

The peptide or composition as described herein is typically administered in an “effective amount”; that is, an amount effective to elicit any one or more inter alia of a therapeutic or prophylactic effect. Persons skilled in the art would be able, by routine experimentation, to determine an effective, non-toxic amount to include in a pharmaceutical composition or to be administered for the desired outcome. In general, the peptide or composition as disclosed herein can be administered in a manner compatible with the route of administration and physical characteristics of the recipient (including health status) and in such a way that it elicits the desired effect(s) (i.e. therapeutically effective and/or protective). For example, the appropriate dosage of a composition may depend on a variety of factors including, but not limited to, a subject's physical characteristics (e.g., age, weight, sex), and other factors that may be recognized by persons skilled in the art. Other illustrative examples of general considerations that may be considered when determining, for example, an appropriate dosage of the compositions are discussed by Gennaro (2000, “Remington: The Science and Practice of Pharmacy”, 20th edition, Lippincott, Williams, & Wilkins; and Gilman et al., (Eds), (1990), “Goodman And Gilman's: The Pharmacological Bases of Therapeutics”, Pergamon Press).

### Kit

Conveniently, peptides according to the invention, such as the four MBP peptides of SEQ ID Nos. 1, 2, 3 and/or 4, may be administered together, in the form of a mixed composition or cocktail. However, there may be circumstances in which it is preferable to provide the peptides separately in the form of a kit, for simultaneous, separate, sequential or combined administration.

For example, the kit may comprise peptides, such as the four peptides of SEQ ID Nos. 1, 2, 3 and 4, in separate containers, or two containers, each comprising two peptides. The contents of the containers may or may not be combined prior to administration.

The kit may also comprise mixing and/or administration means (for example a vapouriser for intranasal administration; or a syringe and needle for subcutaneous/intradermal dosing). The kit may also comprise instructions for use.

Various modifications and variations of the described invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or biology or related fields are intended to be covered by the present invention. All publications mentioned in the above specification are herein incorporated by reference.

### EXAMPLES

### Example 1—Safety and Effect on Immune Tolerance of ATX-MS-1467 in Subjects with Relapsing Multiple Sclerosis

An Open-label, One-arm, Proof of Concept Trial was carried out to evaluate the safety of ATX-MS-1467 (MSC2358825A) and its effect on immune tolerance in subjects with relapsing Multiple Sclerosis.

### Investigator(s)/Study Center(s):

This clinical study was conducted at 8 study sites in total; 7 sites in Russia and 1 in Latvia. The Coordinating Investigator was Natalia N. Maslova, MD, PhD.

### Study Period (Years):

5 Feb. 2014 (first subject screened) to 11 Apr. 2016 (last subject last visit)

### Phase of Development:

### IIa

### Objectives:

The primary objective of the study was to evaluate the effects of ATX-MS-1467 administered intradermally (ID), titrated to a dose of 800 μg every 2 weeks (biweekly), for a total period of 20 weeks on 1.5 tesla (T) magnetic resonance imaging (MRI) parameters compared to a Baseline Control Period off treatment in subjects with relapsing multiple sclerosis (MS).

Secondary objectives of the study were:
To evaluate the effects of ATX-MS-1467 administered ID, titrated to a dose of 800 μg biweekly, for a total period of 20 weeks on other MRI parameters
To evaluate the effects of ATX-MS-1467 administered ID, titrated to a dose of 800 μg biweekly, for a total period of 20 weeks on clinical parameters
To evaluate the safety of ATX-MS-1467 administered ID, titrated to a dose of 800 μg biweekly, for a total period of 20 weeks.

Exploratory objectives of the study were:
To evaluate the maintenance of any effects of ATX-MS-1467 on MRI parameters in responders during a 16-week off-treatment follow-up period
To explore the effect of treatment on disease markers (e.g., oligoclonal bands [OCB], immunoglobulin G [IgG] index, demyelination) in peripheral blood and in CSF over time
To explore immunogenicity (based on serum anti-peptide antibody levels) of ATX-MS-1467 over time.

### Methodology:

This was a multi-center, open-label, Phase IIa study in subjects with relapsing MS. The study consisted of 5 periods; subjects remained in the study for 48 weeks.

Screening Period (4 weeks): Prior to entering the Baseline Control Period, subjects were screened to establish their initial eligibility. Subjects were required to have completed any prior treatment with corticosteroids at least 30 days prior to their first MRI scan at Visit 2. Subjects taking any other non-permitted MS therapy at Visit 1 discontinued all such medications as soon as possible after it had been confirmed they had the human leukocyte antigen (HLA) DRB1*15 genotype and were eligible for the study based on their Visit 2 MRI scan.

Baseline Control Period (8 weeks/3 visits): Subjects who were HLA-positive underwent 3 brain MRI scans (with a minimum interval of 28 days between successive scans) to determine their eligibility based on degree of MRI activity. Following confirmation of subject eligibility with respect to MRI criteria, subjects could have had an optional lumbar puncture for the collection of cerebrospinal fluid (CSF). During the Baseline Control Period, subjects did not receive treatment for MS.

Titration Period (4 weeks/3 visits): Following completion of the Baseline Control Period, eligible subjects entered the Titration Period during which biweekly ATX-MS-1467 ID was titrated from the starting dose (50 μg) to the maximum dose (800 μg) according to the following schedule:
Day 1: ATX-MS-1467 50 μg ID
Day 15: ATX-MS-1467 200 μg ID
Day 29: ATX-MS-1467 800 μg ID.

Treatment Period (16 weeks/8 visits): During the Treatment Period, subjects received biweekly dosing with ATX-MS-1467 800 μg ID for 16 weeks and attended study visits for dosing and safety evaluation at 2-week intervals; additional clinical evaluations, including MRI scans, were performed on 3 occasions during the Treatment Period.

Follow-up Period (16 weeks/4 visits): Following completion of the Treatment Period, subjects entered the 16-week Follow-up Period. Subjects remained off study treatment during this period and were evaluated for the maintenance of any therapeutic effect. The MRI scans were performed on 3 occasions during the Follow-up Period.

Subjects who prematurely withdrew from treatment with ATX-MS-1467, i.e., who withdrew during the Titration or Treatment Periods, entered an 8-week Safety Follow-up Period (2 visits).

### Number of Subjects:

At least 15 evaluable subjects were planned for study participation.

A total of 93 subjects were screened for study participation with 37 (39.8%) subjects enrolled in the study to begin the Baseline Control Period. There were 19 subjects who entered and completed the Titration Period and entered the Treatment Period. All 19 subjects treated were included in the Intention-to-Treat (ITT), Modified ITT (mITT), and Safety Analysis Sets.

### Diagnosis and Main Criteria for Inclusion:

Male and female outpatients, 18 to 65 years of age, inclusive, who had relapsing-remitting MS (RRMS) or secondary progressive MS (SPMS) according to the McDonald diagnostic criteria (2010), clinical evidence of recent MS activity, at least 1 contrast-enhanced lesion (CEL) on MRI at Visit 2, and the presence of at least 1 new CEL from Visit 2 to Visit 4 were eligible for study participation. Subjects had to have an Expanded Disability Status Scale (EDSS) score between 0 and 5.5, be HLA DRB1*15-positive, and be neurologically stable prior to the start of study treatment. Subjects were not eligible for this study if they had primary progressive MS, renal conditions that precluded the administration of gadolinium (Gd), lymphocyte count <500/μL or neutrophil count <1500/μL at pretreatment visits, or other underlying medical conditions that precluded participation in the study.

Previous treatments with beta-interferon, plasma exchange, or intravenous gamma globulin within the 8 weeks prior to Study Day 1; steroids or adrenocorticotropic hormone within the 30 days prior to the Visit 2 MRI scan; or glatiramer acetate, cytotoxic agents, fingolimod, laquinimod, teriflunomide, total lymphoid irradiation, stem cell or bone marrow transplantation, monoclonal antibody therapy, dimethyl fumarate, dirucotide, any disease related T-cell vaccine or peptide-tolerizing agent for the treatment of MS were not allowed.

### Test Product(s): Dose and Mode of Administration, Batch Number(s):

The investigational medicinal product was ATX-MS-1467 and was administered ID biweekly for a total of 20 weeks, from a starting dose of 50 μg and titrated over 4 weeks to the final dose of 800 μg.

Individual batch numbers are available upon request.

### Duration of Treatment:

From Visit 1 to Visit 19, the maximum duration of study participation was 48 weeks. The duration of study treatment was 20 weeks.

### Criteria for Evaluation:

### Efficacy:

The primary endpoint was the change in the average number of T1 CEL at the last 3 on-treatment scans (Weeks 12, 16, and 20) compared to the average number of T1 CEL at the 3 baseline scans (Visits, 2, 3, and 4).

Secondary endpoints included the following:
MRI
   Total number of T1 CEL at each scheduled post-baseline MRI visit
   Change from baseline (average of the 3 baseline scans, Visits 2, 3, and 4) in total number of T1 CEL at each scheduled post-baseline MRI visit
   Change from baseline (average of the 3 baseline scans, Visits 2, 3, and 4) in total volume of T1 CEL at each scheduled post-baseline MRI visit
   Total number of new or newly enlarging T2 lesions at each scheduled post-baseline MRI visit
   Change from Visit 4 in total number of T1 CEL at each scheduled post-baseline MRI visit
   Change from Visit 4 in total volume of T1 CEL at each scheduled post-baseline MRI visit.
Clinical
   Mean annualized relapse rate (ARR) at Week 20
   Time to first relapse
   Change from baseline in total EDSS score at Week 20
   Change from baseline in total Multiple Sclerosis Functional Composite (MSFC) score at Week 20.

The following exploratory endpoints were also considered:
Maintenance of the effects of ATX-MS-1467 on MRI parameters following discontinuation of treatment at Weeks 28 and 36 in the subgroup of responders
Serum anti-peptide antibody levels at Weeks 11, 20, and 24

### Safety:

Safety endpoints included the following:
Nature, frequency, and severity of treatment-emergent adverse events (TEAEs)
Frequency and severity of injection site reactions
Vital sign measurements, physical examination findings, clinical laboratory variables, electrocardiograms, and the frequency and timing of premature termination from the study.

### Statistical Methods:

The sample size for this study was based on 3 assumptions: (1) A treatment effect of 70% reduction in the number of CEL as compared to the average number of CEL at the 3 baseline scans; (2) the mean number of CEL at baseline was 5 with a standard deviation (SD) of 6; and (3) the mean number of CEL during the post-treatment period (Weeks 24 to 36) was 1.5 with a SD of 1.8. Using a 2-sided 5% level, >80% and >90% of the simulated studies showed a statistically significant result with sample sizes of 12 and 14 subjects, respectively. Thus, a sample size of 15 subjects was selected.

Analysis of the primary and secondary endpoints was based on the mITT Population, except for evaluation of the maintenance of response which was performed using the Responders Population and safety analyses which were based on the Safety Population. Responders were defined as those subjects in whom there was a reduction from baseline (average of the 3 baseline scans at Visits 2, 3, and 4) of 60% in the number of T1 Gd enhancing lesions at Week 20 (average of the last 3 on-treatment scans at Weeks 12, 16, and 20).

The primary endpoint was analyzed using a nonparametric Wilcoxon Signed Rank test for a test of shift in location due to the treatment effect based on an exact distribution of the signed rank statistic where the distribution is a convolution of scaled binomial distributions. A supportive analysis was performed to estimate mean percentage reduction in new T1 Gd-enhancing lesions during the treatment period compared to baseline control period using generalized estimating equations (GEE) linear regression model with negative binomial and Poisson link functions.

The same nonparametric procedure used for the primary efficacy endpoint was carried out based on new T1 Gd-enhancing lesions. Descriptive statistics were displayed for secondary endpoints at each applicable visit for the mITT Analysis Set.

Continuous variables were summarized descriptively using the number of observations, means, SD, 95% confidence interval (CI), median, minimum, and maximum. Categorical variables were summarized using frequency counts and percentages. Time to event variables were presented as Kaplan Meier plots, median, and 95% Cl.

### Summary and Conclusions:

### Subject Disposition:

There were 93 subjects screened for study participation; 37 (39.8%) subjects were enrolled. Nineteen (51.4%) subjects entered and completed the Titration Period and 18 (48.6%) subjects completed the Treatment Period. One subject (2.7%) discontinued investigational medicinal product (IMP) due to an adverse event (AE) of diarrhea. A second subject (2.7%) withdrew consent after completing the Treatment Period. All 19 eligible subjects were included in the ITT, mITT, and Safety Analysis Sets. Seven subjects who demonstrated a reduction from baseline in the number of T1 Gd enhancing lesions of ≥60% at Week 20 were included in the Responders Analysis Set.

### Demographics and Baseline Characteristics:

The mean age of subjects in the study was 27.1 years (range 19 to 38 years) with the majority of subjects <30 years of age (73.7%). Most subjects were female (78.9%) and all subjects were white. All 19 subjects had a diagnosis of RRMS with the majority of subjects (89.5%) reporting 1 to 2 relapses in the 24 months prior to Visit 2. The median EDSS score at baseline was 2.00 (range 1.5 to 3.5). The median MSFC score at baseline based on the National Multiple Sclerosis Society (NMSS) reference population was 0.470 (range −0.95 to 1.21). The mean number and volume of T1 Gd-enhancing lesions were 7.4 (range 1 to 31) and 0.838 mL (range 0.05 to 3.65 mL), respectively.

### Efficacy Results:

There was a statistically significant decrease in the average number of T1 Gd-enhancing lesions on treatment (Weeks 12, 16, and 20) compared with baseline based on a nonparametric analysis using the mITT analysis set (p=0.0143). The Hodges-Lehmann estimate of location shift (95% CI) was −1.3 (−6.3, 0.0). Similarly, there was a statistically significant decrease in average number of new T1 Gd-enhancing lesions (p=0.0106). The Hodges-Lehmann estimate of location shift (95% CI) was −1.3 (−5.7, 0.0). The results of supportive analysis of new T1 Gd-enhancing lesions are consistent with the primary analysis.

Numerical decreases from baseline in mean T1 Gd-enhancing lesion count and volume were noted at all post-dose assessments. The mean number of lesions at baseline was 7.4 (range 1 to 31) and mean change from-baseline in lesion count ranged from −4.6 to −1.6. The mean lesion volume at baseline was 0.838 mL (range 0.05 to 3.65 mL) and the mean change from baseline in lesion volume ranged from −0.579 to −0.225 mL. Similarly, numerical decreases in mean T1 Gd-enhancing lesions and volumes from Week 0 to each post-dose assessment were noted. The mean change from Week 0 in lesion count ranged from −3.5 to −0.9 and the mean change from Week 0 in lesion volume ranged from −0.473 to −0.157 mL. The median number of new T1 Gd-enhancing lesions was similar from Week 12 (1.5) through the end of study (ranging from 0.0 at Week 28 to 2.0 at End of Study). The median number of new/enlarging T2 lesions decreased from 8.0 (range 0 to 89) at Week 12 to 1.0 at Week 16 (range 0 to 20), and the median count ranged from 1.0 to 3.0 at subsequent visits.

During study treatment, a single relapse occurred in 3 (15.8%) subjects; no relapse during treatment was reported for the remaining 16 subjects. The estimated mean ARR was 2.60. For these 3 subjects, the onset of relapse occurred on Days 50, 59, and 89 and the Kaplan-Meier estimates of the probability of not experiencing relapse decreased from 1.00 at Week 4 to 0.84 at Week 20.

The change from baseline to the End of Treatment visit in EDSS score was not statistically significant. Similarly, the changes from baseline to the End of Treatment visit in MSFC scores although not statistically significant showed a strong trend towards improvement when using values from the NMSS Task Force Database as reference.

### Safety Results:

Overall, 78.9% of subjects reported at least 1 TEAE during study conduct; TEAEs in 57.9% of subjects were assessed as related to IMP. The most frequently-reported TEAEs were injection site erythema (26.3%), headache (21.1%), and nasopharyngitis (15.8%).

There were no deaths, serious TEAEs, or TEAEs of severe intensity reported. One subject discontinued IMP due to a TEAE of diarrhea, which was prolonged in duration and assessed as moderate in intensity and related to IMP.

The onset of TEAEs for most subjects was <26 days from the start of IMP.

All injection site reactions (36.8% of subjects) during the study were of mild intensity and the most frequently-reported symptoms were erythema, pruritus, and induration.

Three subjects had a 7% decrease in weight during the study; otherwise, there were no clinically relevant changes in clinical laboratory, vital sign, or electrocardiogram parameters.

### Conclusions:

The median decrease in T1 Gd-enhancing lesions during the treatment period compared to the baseline control period was statistically significant (p=0.0143) based on a nonparametric Wilcoxon signed rank test. The Hodges-Lehmann estimate of location shift (95% CI) was −1.3 (−6.3, 0.0).

Supportive analyses using negative binomial and Poisson GEE models yielded consistent findings. Compared to the baseline control period, the mean percentage reduction in new T1 Gd-enhancing lesions during the treatment period was statistically significant (p value=0.0109). The GEE-estimated mean percentage reduction (95% CI) using the negative binomial model was 38.4% (10.6%, 57.6%).

Numerical decreases from baseline and from Week 0 in mean T1 Gd-enhancing lesion count and volume were noted at all post-dose assessments.

The median number of new/enlarging T2 lesions decreased from 8.0 at Week 12 to 1.0 at Week 16, and ranged from 1.0 to 3.0 at subsequent visits.

Few subjects (15.8%) experienced relapse during the study. The Kaplan-Meier estimated probability of not experiencing relapse by Week 20 is 0.84.

No statistically significant changes from baseline to the End of Treatment visit in EDSS or MSFC scores were noted although there was a strong trend towards improvement in MSFC (P=0.0542).

The MSFC results obtained as a secondary endpoint as described above were then further analyzed using the Wilcoxon matched-pairs signed rank test. It was found that a significant (P=0.010) improvement in cognition underpinned the strong trend towards overall disability reduction measured using MSFC score. These results are shown in FIG. 2. It was very unusual to see any improvement after only 6 months.

No safety or tolerability concerns were identified following treatment with ATX-MS-1467 800 μg administered ID every 2 weeks.

### Example 2—Treatment with ATX-MS-1467 Persistently Triggers IL-10 but not Pro-Inflammatory Cytokine Release

### Methods

Double transgenic heterozygous mice, referred to here as DR2/Ob1Het/Het, were used for these studies. These mice express human leukocyte antigen (HLA) isotypes DRA*0101 and DRB1*1501 under the mouse major histocompatibility (MCH)-II promoter and the MBP84-102-specific TCR (Ob.1A12) expressed under mouse TCRα and β promoter/enhancer elements.

DR2/Ob1Het/Het mice were treated and/challenged by a single or multiple subcutaneous (s.c.) injections of 100 μg of ATX-MS-1467 or 25 μg of a HLA binding protein (HLAbp) unrelated to EAE and/or 30-1000 μg of myelin basic protein (MBP, Sigma, M1891). Treatment paradigms varied between studies and groups (see specifics in each study). Chronic treatment with ATX-MS-1467 or with HLAbp followed a 3× weekly regimen.

Cytokine levels were quantified in serum of DR2/Ob1Het/Het mice at different time points using a Milliplex MAP mouse Cytokine/Chemokine magnetic kit (MCYTOMAG-70-PMX). For simplicity, only four representative cytokines are shown in FIG. 4.

Leukocyte activation gene 3 (Lag3) expression was assessed in a MACSQuant analyzer gating on CD4+ spleen lymphocytes from DR2/Ob1Het/Het mice that had been immunized with an emulsion containing spinal cord homogenate (SCH)/complete Freund's adjuvant (CFA) and treated with either ATX-MS-1467 (100 μg, 3×/week) or vehicle between 4-14 days post-immunization.

### Results

It was found that acute treatment of DR2/Ob1Het/Het mice with MBP (FIG. 4) or ATX-MS-1467 (FIG. 5) induces the secretion of both pro and anti-inflammatory cytokines in the blood.

FIG. 5 also shows that cytokine release is transient, peaking at 2 h and returning to baseline values 4-24 after the treatment.

These data suggest that there is a pool of T-cells in DR2/Ob1Het/Het mice that are ready to react with the MBP, and with at least one of the peptide sequences that are present in ATX-MS-1467.

A single injection of ATX-MS-1467 induces secretion of IL-2, IL-17, and IFN-g in the blood, but pro-inflammatory cytokine release is reduced following subsequent dosing (3×/week). Conversely, even with repeat dosing, ATX-MS-1467 continues to induce secretion of the anti-inflammatory cytokine IL-10.

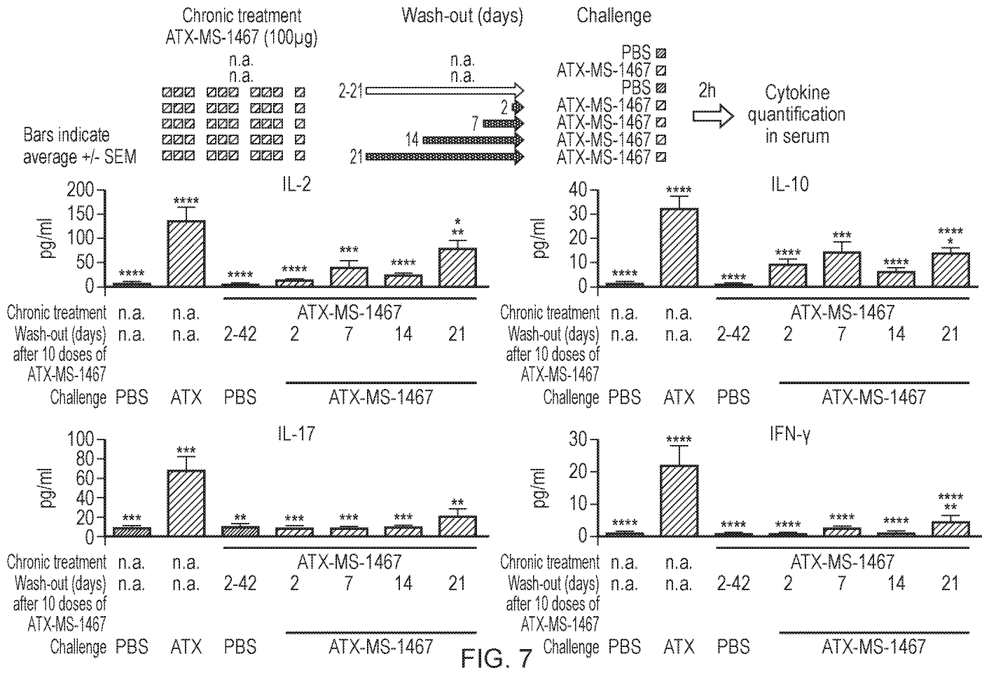

As shown in FIG. 7, cytokine secretion after a new ATX-MS-1467 challenge following a wash-out period of up to 21 days in mice that had received chronic treatment was minor in comparison with the response in acutely treated mice. Nevertheless, there was a statistically significant release of IL-2 and IFN-γ on the 3 week wash-out group point revealing a small but significant loss of tolerance.

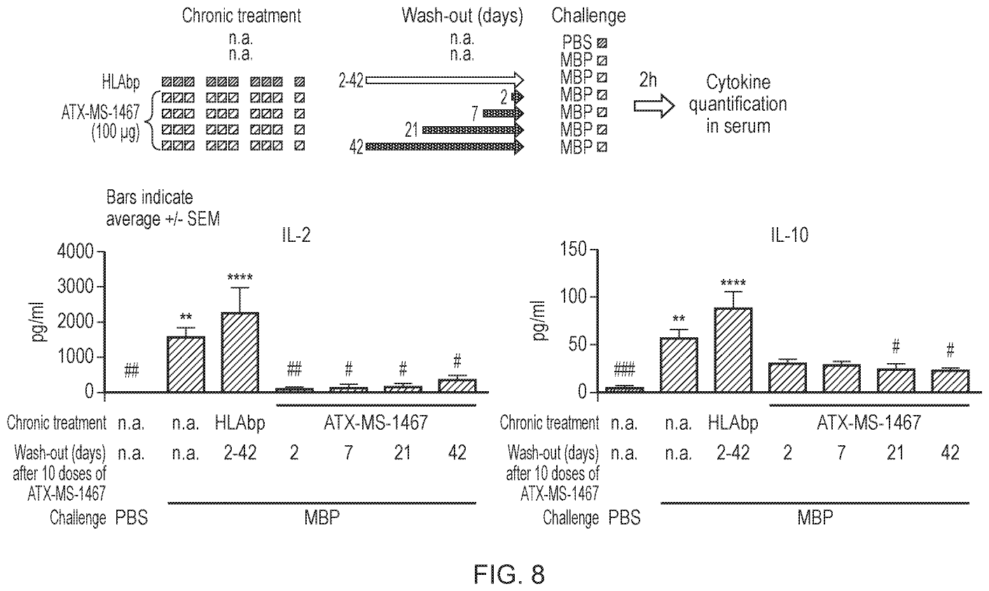

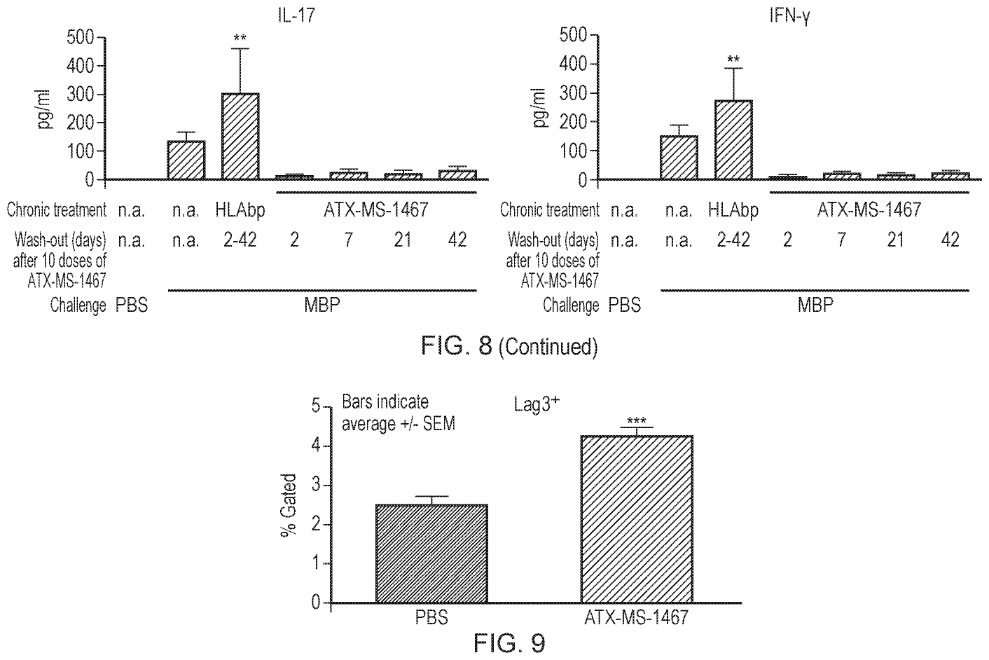

FIG. 7 suggests a prolonged tolerogenic effect of ATX-1467. In FIG. 8 we challenged mice with full length MBP after they had received chronic treatment with ATX-MS-1467 followed by a wash-out period lasting from 2-42 days. Chronic treatment with ATX-MS-1467 followed by a challenge with full length MBP also shows a tolerizing effect against MBP, even if the challenge was done after a wash-out period of up to 6 weeks. Replacement of ATX-MS-1467 by a HLA-binding protein that is unrelated to MBP, fails to induce tolerance, demonstrating the antigen-specificity of the effect.

Lag3 is a cell surface molecule known to interact with MHC-II, to act as an intrinsic inhibitory molecule in T lymphocytes, to be expressed on a population of IL-10-secreting induced Tregs and to play a role in the expression of tolerance. The increase in the frequency of Lag3-expressing CD4+ spleen lymphocytes (FIG. 9) along with our previous demonstration of the increase in IL-10 secretion from splenocyte cultures of mice treated with ATX-MS-1467 suggests that the induction of Tregs is a likely mechanism of action of ATX-MS-1467.

### Conclusions

The data show that chronic treatment with ATX-MS-1467 shifts the cytokine response in blood to a long-lasting tolerized state.

This state is characterized by a pattern of low or virtually no pro-inflammatory cytokine release despite detectable IL-10 production following an antigen-specific challenge.

It is reasonable to expect that T cells bearing a specificity comprised within the epitope sequences that were tolerized by the treatment, once exposed to their cognate antigen in the CNS would display a similar pattern of cytokine secretion, highlighting beneficial therapeutic implications.

### Example 3—ATX-MS-1467 Halts Disease Progression and Reduces Central Nervous System Inflammation

### Methods

In Lewis rats, experimental autoimmune encephalomyelitis (EAE) was induced using an emulsification of ATX-MS-1467 and Complete Freund's Adjuvant (CFA) on Day 0. Rats also received pertussis toxin injections on Days 0 and 2.

In double-transgenic (DTg; human HLA-DR15/MBP-specific T-cell receptor) ‘humanized’ mice, EAE was induced using an emulsification of spinal cord homogenate (SCH) and CFA on Day 0. Mice also received a pertussis toxin injection on Days 0 and 2.

Throughout the study, neurological deficits were measured using a standardized clinical score scale: 0=no clinical signs, 1=limp tail, 2=impaired righting reflex, 3=partial hind limb paralysis, 4=complete hind limb paralysis, 5=moribund/death.

If animals reached a score of 4 they were euthanized to reach a humane endpoint.

Rats were treated prophylactically either with subcutaneous (sc) phosphate-buffered saline (PBS) vehicle or ATX-MS-1467 (n=10 per treatment group) starting 3 weeks before EAE induction. Mice were treated prophylactically (starting on the day of induction) or therapeutically (starting 7 days post-induction) with subcutaneous vehicle, ATX-MS-1467, MBP82-98 (dirucotide), or glatiramer acetate (GA [Copaxone®, Teva Neuroscience, Inc., North Wales, Pa., USA]; n=9-10 per treatment group).

ATX-MS-1467 was tested head-to-head with MBP82-98 and GA. MBP82-98 is a 17-amino acid synthetic peptide identical to MBP and GA is a random polymer of four amino acids found in MBP. For both compounds, the human equivalent dose (HED) was calculated based on body surface area: MBP82-98=12 μg/dose and GA=75 μg (3.75 mg/kg)/dose.

To investigate the mechanism of action, cellular infiltrates were examined using multicolor flow cytometry in samples from DTg EAE mice dosed either with vehicle or ATX-MS-1467 (100 μg once/week) starting at Day 0. Mice were euthanized on Day 15 during the peak of disease (n=6 per treatment group) and spinal cord/brains were harvested for analysis. Briefly, after exclusion of doublets and dead cells (propidium iodide staining), macrophages were differentiated from microglia (both CD11b+ and CD45+) via the level of expression of CD11b high and low, respectively. Lymphocyte subpopulations (GR1−) were defined using CD19 (B cells), CD4, and CD8 markers (T cells).

Statistical analyses for significant differences on clinical scores by treatment over time and cumulative clinical scores (area under the curve) were performed using Kruskal-Wallis with Dunn's post hoc analysis of comparisons. Disease onset was analyzed by Kaplan-Meier curve and significance was determined using the log-rank (Mantel-Cox) test. Cellular brain infiltrates were analyzed by Student's t-test. Statistical significance was considered at *p, 0.05, *p, 0.01, and ***p, 0.001.

### Results

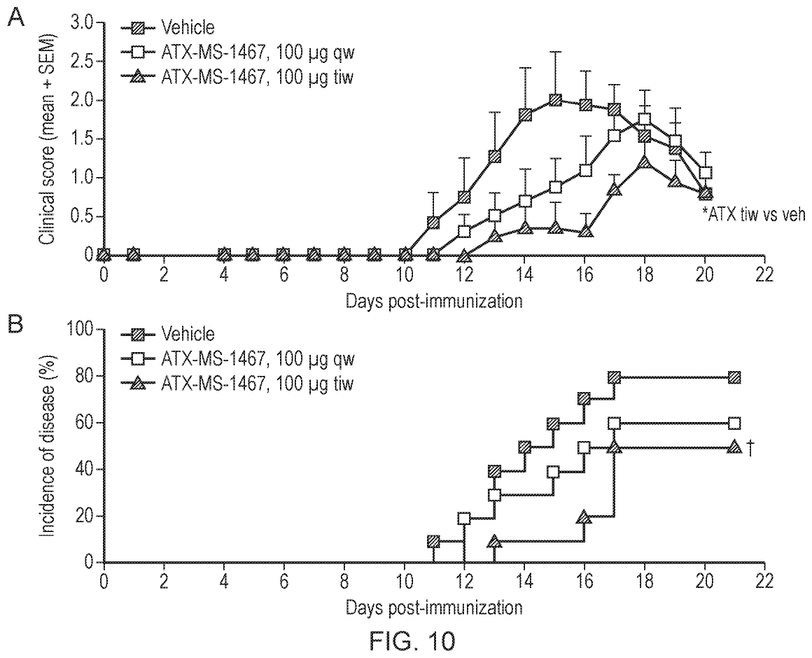

Lewis rats were treated either with vehicle or with ATX-MS-1467 (100 μg/dose, sc) once weekly or three times weekly (n=10 per group), starting 3 weeks before EAE induction. Dosing with ATX-MS-1467 three times weekly significantly reduced disease severity compared with vehicle treatment (FIG. 10A). In addition, disease onset was significantly delayed in rats dosed three times weekly with ATX-MS-1467 compared with vehicle (FIG. 10B).

### DTg Humanized Mouse Model of EAE

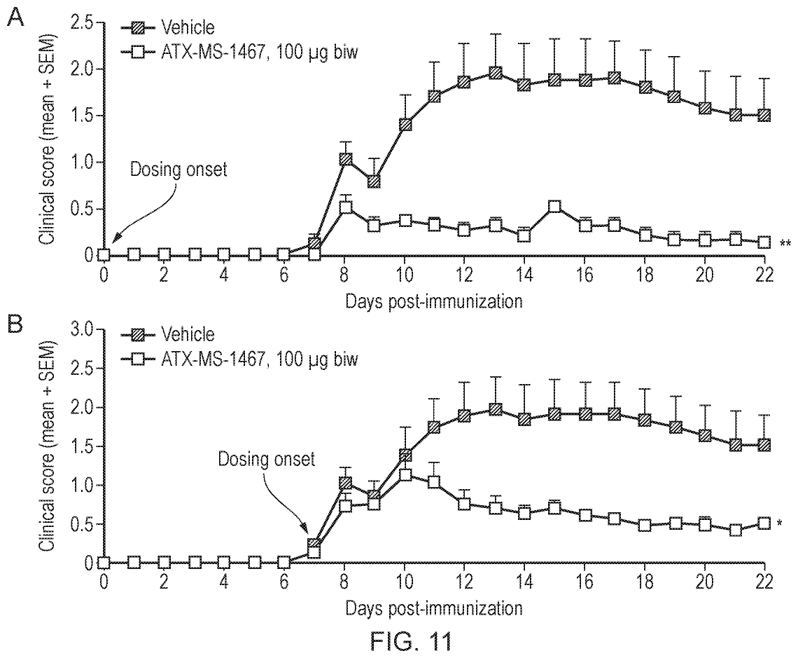

Pre-treatment (starting on day of immunization, Day 0) or therapeutic dosing (starting on Day 7 post-immunization) with ATX-MS-1467 (100 μg/dose, sc) reduced SCH-induced EAE in DTg humanized mice. The disease severity was significantly reduced by twice-weekly ATX-MS-1467 treatment versus vehicle-treated controls (FIG. 11A).

Twice-weekly treatments with ATX-MS-1467 also significantly reduced disease severity compared with vehicle even though treatment was initiated after the first signs of paralysis had developed (FIG. 11B).

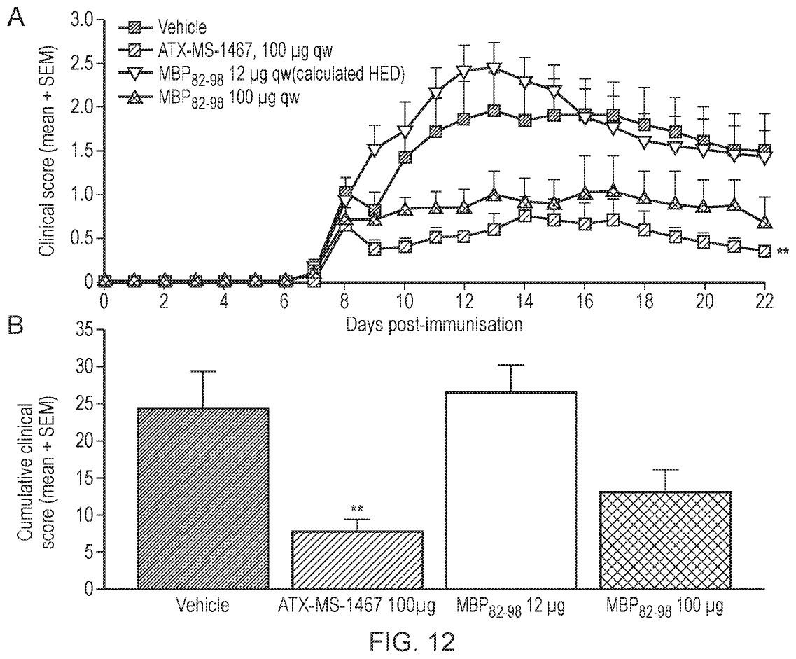

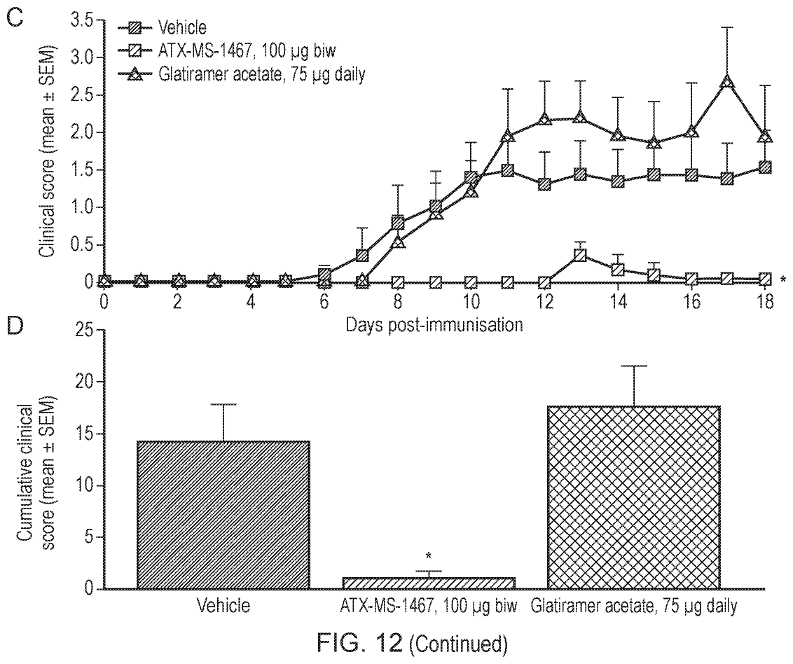

Once-weekly dosing with ATX-MS-1467 (100 μg/dose, sc) significantly reduced disease severity compared with vehicle treatment, but the same dosing regimen with MBP82-98 (12 μg or 100 μg/dose) did not show significant effects in this study (FIGS. 12A, 12B).

In a separate study, treatment with ATX-MS-1467 (100 μg/mouse, sc) twice weekly starting on Day 0 significantly reduced disease severity compared with daily treatment either with vehicle or GA at the calculated HED (75 μg/dose, sc; FIGS. 12C, 12D).

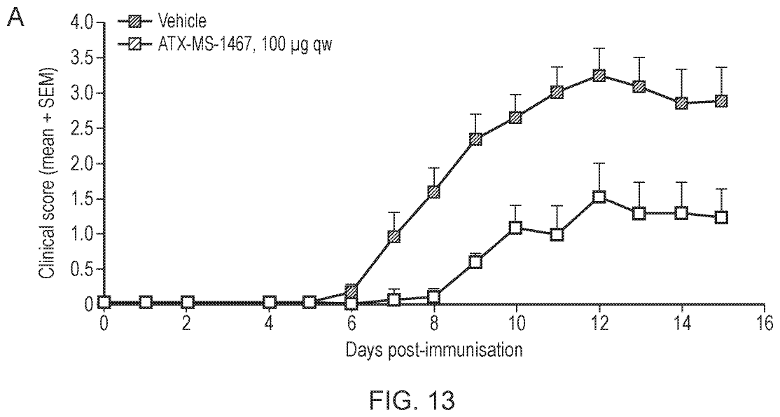

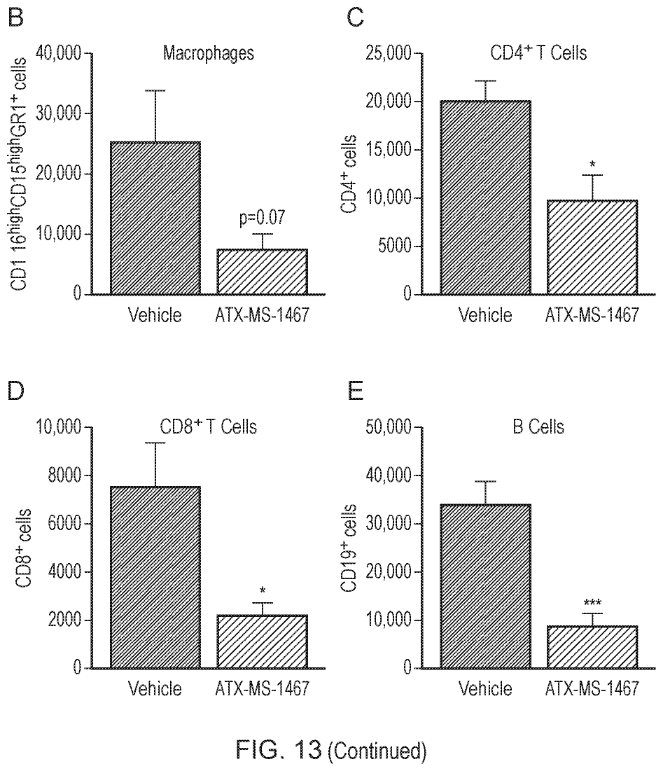

Treatment with ATX-MS-1467 (100 μg/dose once weekly) starting on the day of immunization (Day 0) reduced EAE-induced inflammatory cell infiltration of the central nervous system (CNS), which was associated with reduced clinical score (FIG. 13A).

On Day 15, mice were euthanized and brain and spinal cords were harvested for analysis of brain infiltrates using multicolored flow cytometry. The numbers of macrophages, T cells, and B cells were significantly lower in mice treated with ATX-MS-1467 compared with vehicle treated mice (FIGS. 13B to 13E).

### Example 4—ATX-MS-1467 Ameliorates Pathological Changes and Inhibits Cytokine Production in a Mouse Model of Multiple Sclerosis

### Methods

### Disease Induction

Disease was induced in DR2/Ob1het/het mice by a subcutaneous injection of an adjuvant emulsion containing syngeneic spinal cord homogenate (SCH). Clinical disability was measured with a subjective 0-5 scale.

### Treatment

ATX-MS-1467 was injected subcutaneously at 3, 10, 30 or 100 μg/mouse 3×/week starting 0, 7 or 14 days post immunization (dpi), depending on the study.

### Histological Analysis

For each mouse 10 spinal cord sections were stained via hematoxylin and eosin (H&E) for inflammation or Luxol fast blue (LFB) staining for assessment of myelin content, or immunoreactivity against CD3 and CD45R immunoreactivity for T or B cells respectively. The sections were scanned (Nanozoomer 2.0 HT) and semi-quantitatively analyzed in a blinded fashion. A similar scale was applied for HE, CD3 and CD45B stainings except that the interest cells were either hematoxylin stained nuclei, CD3+ or CD45+ cells. H&E, CD3 and CD45B scale: 0=no immune cell infiltration, 1=Immune cells lining meninges, 2=+Perivascular cells, 3=+small multifocal white matter (WM) infiltrates, 4=+multiple and extensive WM infiltrates, 5=Gray matter infiltrates. For demyelination, the dorsal, ventral, right and left lateral WM were individually scored then values were added. LFB scale: 0 (no demyelination), 0.5 (the demyelinated area (DA) is ≤10%), 1 (>10% DA≤20%), 2 (>20% DA≤40%), 3 (>40% DA≤60%), 4 (>40% DA≤80%), 5 (>80% DA≤100%).

### Magnetic Resonance Imaging

Conventional T1-weighted gadolinium-enhanced (Gd+) and T2-weighted multislice MRI sequences were used to examine the effects of ATX-MS-1467 on blood brain barrier (BBB) leakage and lesion development, respectively. BBB leakage was evaluated within 10 minutes of Gd+injection; use of later time points did not indicate further enhancement.

### Splenocyte Culture

Splenocytes from SCH-immunized mice injected with either ATX-MS-1467 or phosphate buffered saline (PBS) 3×/weekly from dpi0-dpi7 were harvested and stimulated in the presence of ATX-MS-1467 for 48 or 72 hours (h) at which times supernatants were collected for cytokine quantification by ELISA. Cell proliferation was assessed by partial replacement of the supernatant with a 3H-Thymidine solution, posterior incubation for 8 h followed by quantification of the radioactivity in the cells.

### Results

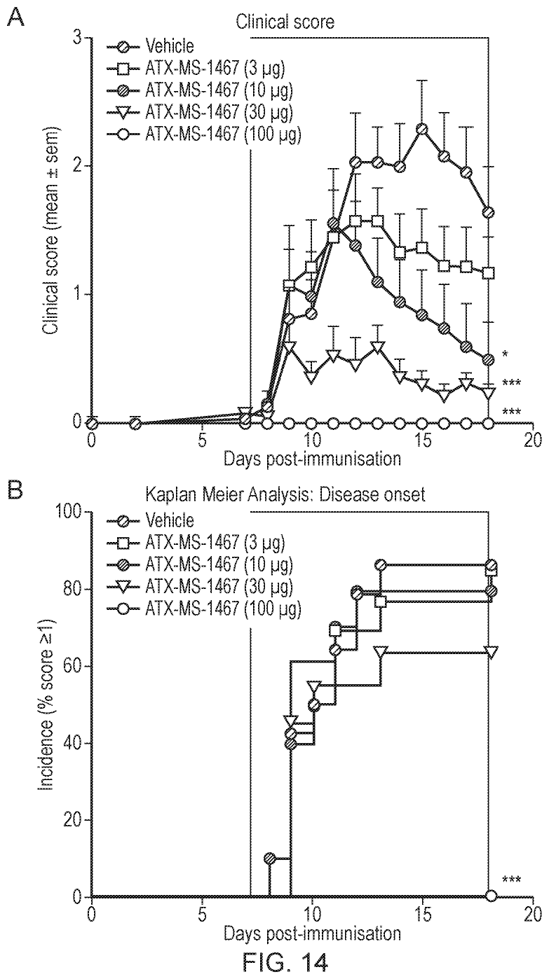

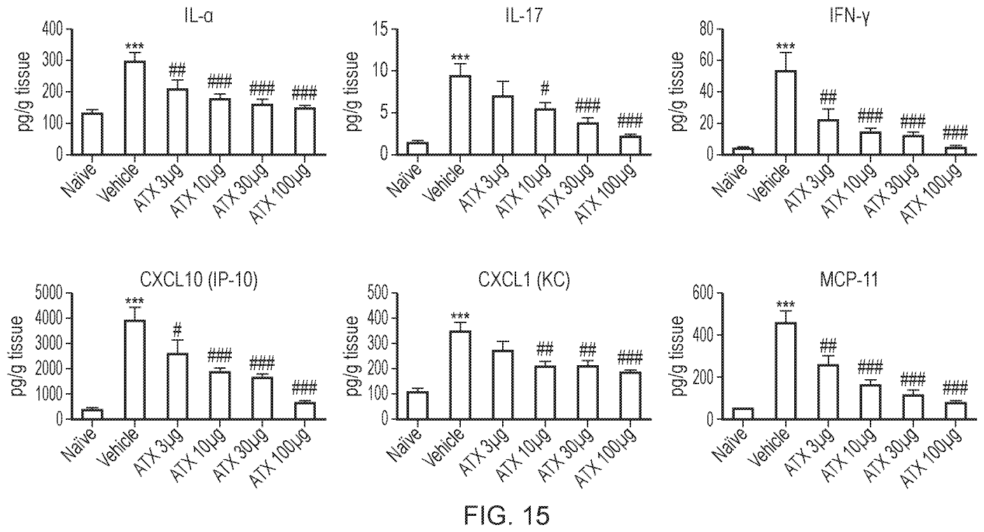

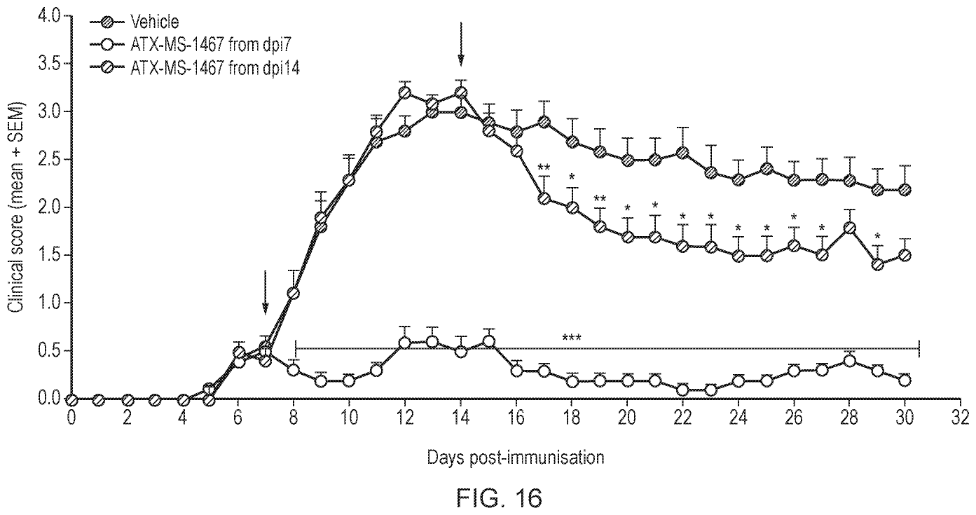

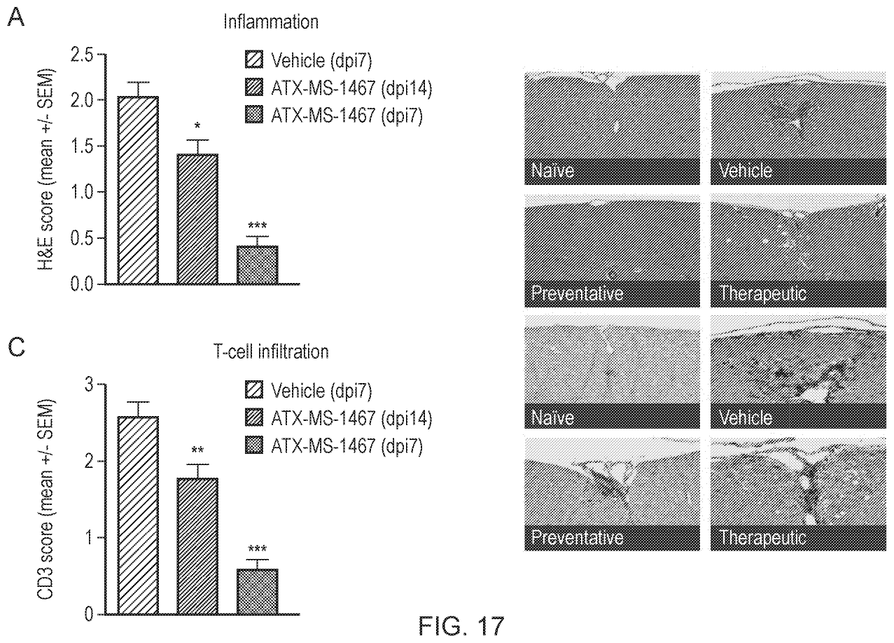

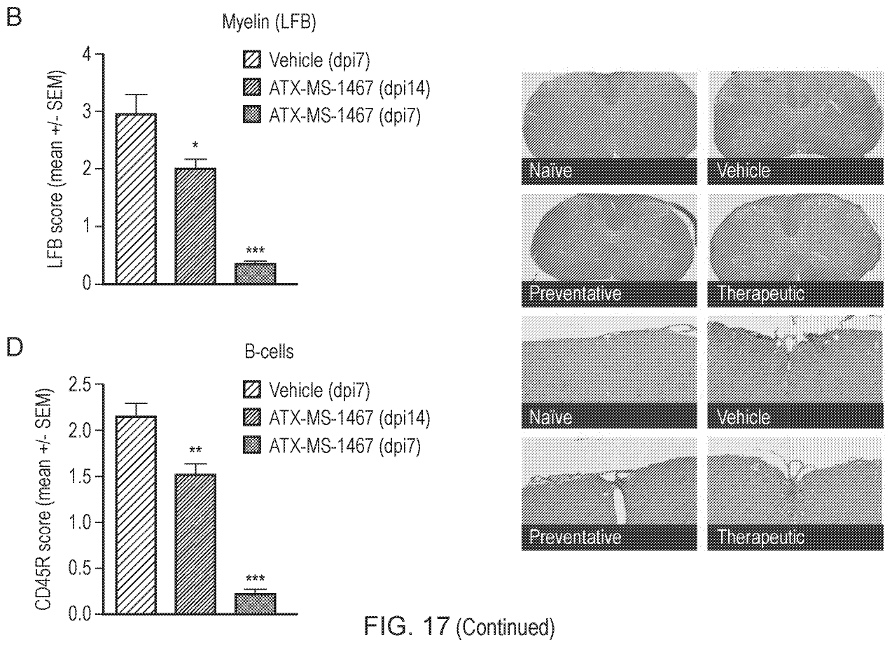

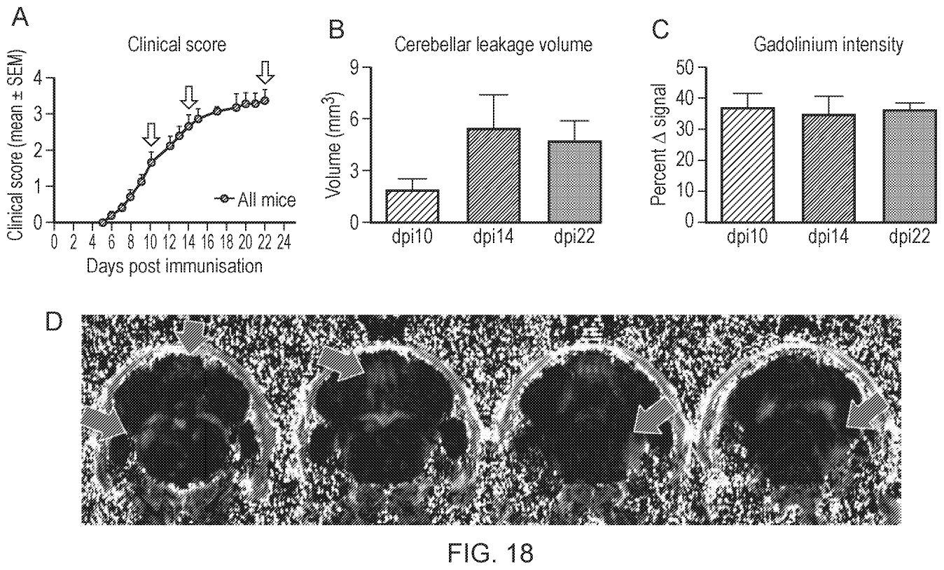

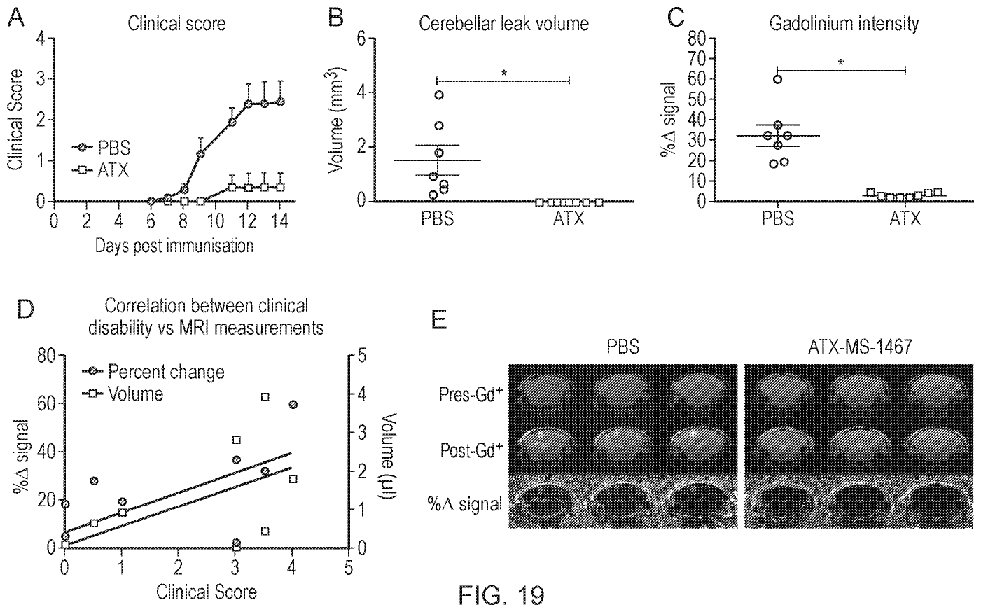

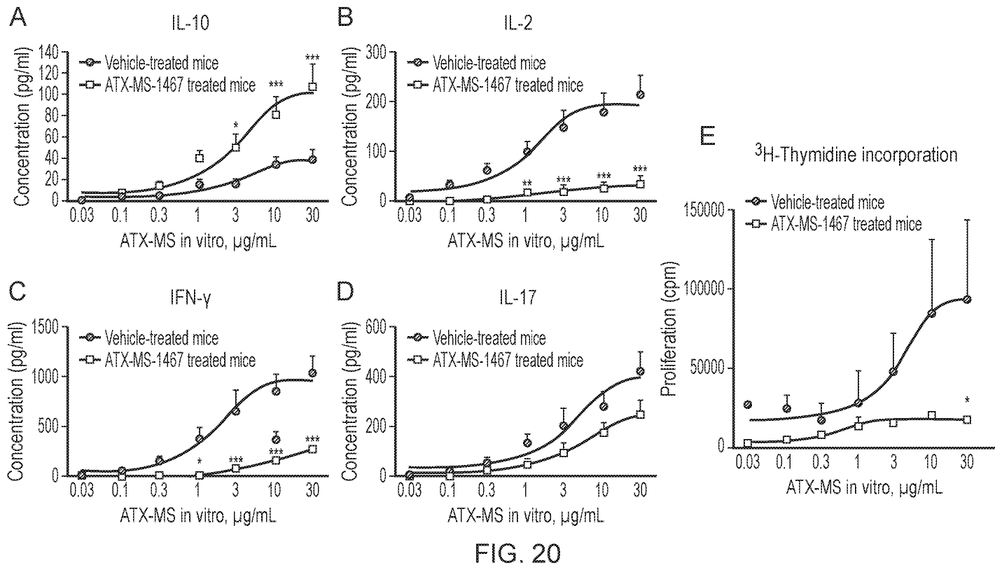

See FIGS. 14 to 20. It was found that treatment with ATX-MS-1467 dose-dependently inhibited severity and/or prevented onset of SCH-induced EAE in DR2/Ob1het/het mice. Those effects were associated with reduced concentrations of pro-inflammatory cytokines at the central nervous system (CNS).

The therapeutic effect of ATX-MS-1467 was observed even when treatment was initiated after peak of disease and was confirmed via pathological analysis of the spinal cord for the assessment of inflammation, T- and B-cell infiltration and myelin damage.

Prophylactic treatment with ATX-MS-1467 prevented BBB leakage in humanized model of MS, as measured by T1-weighted Gd+MRI.

The MRI read-outs and data were consistent with Phase1b data in humans suggesting that such pre-clinical endpoints may provide predictive information for clinical studies.

Cell proliferation and cytokine secretion data from the splenocytes of mice treated with ATX-MS-1467 suggested that its mechanism of action may involve increased synthesis of the anti-inflammatory cytokine IL-10 and inhibition of the synthesis of IL-2 and IFN-γ.

1. A method for treating or preventing cognitive impairment in a subject comprising administering to the subject a peptide derived or derivable from a component of myelin selected from myelin basic protein (MBP), myelin oligodendrocyte glycoprotein (MOG) and myelin proteolipid protein (PLP).

2. The method according to claim 1 wherein said subject has a demyelinating disease.

3. The method according to claim 2 wherein said subject has multiple sclerosis, Alzheimer's disease or Parkinson's disease.

4. The method according to any one of claims 1 to 3 wherein said peptide is selected from SEQ ID Nos. 1, 2, 3 and 4.

5. The method according to any one of claims 1 to 4 wherein peptides of SEQ ID Nos. 1, 2, 3 and 4 are administered to said subject.

6. The method according to any one of claims 1 to 3 wherein said peptide is selected from SEQ ID Nos. 7, 8, 9 and 10.

7. The method according to any one of claims 1 to 3 and 6 wherein peptides of SEQ ID Nos. 7, 8, 9 and 10 are administered to said subject.

8. The method according to any one of claims 1 to 3 wherein said peptide is selected from SEQ ID Nos. 12, 16, 18, 23, 24, 25, 26, 27, 28, 29, 30 and 31.

9. The method according to any one of claims 1 to 8 wherein said treatment leads to an improved PASAT score for said subject.

10. A peptide as defined in any one of claims 4 to 8 for use in treating or preventing impaired cognition in a subject.

11. Use of a peptide as defined in any one of claims 4 to 8 in the manufacture of a medicament for treating or preventing impaired cognition in a subject.

12. The peptide for use according to claim 10 or use of a peptide according to claim 11 wherein said subject has a demyelinating disease.

13. The peptide for use or use of a peptide according to claim 12 wherein said subject has multiple sclerosis, Alzheimer's disease or Parkinson's disease.

14. The peptide for use or use of a peptide according to any one of claims 11 to 13 wherein said treatment leads to an improved PASAT score for said subject.

15. A kit for use in treating or preventing impaired cognition in a subject, wherein said kit comprises a peptide as defined in any one of claims 4 to 8.

16. A method for treating or preventing dementia in a subject comprising administering to the subject a peptide derived or derivable from a component of myelin selected from myelin basic protein (MBP), myelin oligodendrocyte glycoprotein (MOG) and myelin proteolipid protein (PLP).

17. The method according to claim 16 wherein said subject has Alzheimer's disease.

18. The method according to claim 16 wherein said subject has Parkinson's disease.

19. The method according to any one of claims 16 to 18 wherein said peptide is selected from SEQ ID Nos. 1, 2, 3 and 4.

20. The method according to any one of claims 16 to 19 wherein peptides of SEQ ID Nos. 1, 2, 3 and 4 are administered to said subject.

21. The method according to any one of claims 16 to 18 wherein said peptide is selected from SEQ ID Nos. 7, 8, 9 and 10.

22. The method according to any one of claims 16 to 18 and 21 wherein peptides of SEQ ID Nos. 7, 8, 9 and 10 are administered to said subject.

23. The method according to any one of claims 16 to 18 wherein said peptide is selected from SEQ ID Nos. 12, 16, 18, 23, 24, 25, 26, 27, 28, 29, 30 and 31.

24. A peptide as defined in any one of claims 19 to 23 for use in treating or preventing dementia in a subject.

25. Use of a peptide as defined in any one of claims 19 to 23 in the manufacture of a medicament for treating or preventing dementia in a subject.

26. The peptide for use according to claim 24 or use of a peptide according to claim 25 wherein said subject has Alzheimer's disease.

27. The peptide for use according to claim 24 or use of a peptide according to claim 25 wherein said subject has Parkinson's disease.

28. A kit for use in treating or preventing dementia in a subject, wherein said kit comprises a peptide as defined in any one of claims 19 to 23.

29. A method for treating or preventing demyelination in a subject comprising administering to the subject a peptide derived or derivable from a component of myelin selected from myelin basic protein (MBP), myelin oligodendrocyte glycoprotein (MOG) and myelin proteolipid protein (PLP).

30. The method according to claim 29 wherein said subject has a neurodegenerative disease.

31. The method according to claim 29 wherein said subject has multiple sclerosis, Alzheimer's disease, or Parkinson's disease.

32. The method according to any one of claims 29 to 31 wherein said peptide is selected from SEQ ID Nos. 1, 2, 3 and 4.

33. The method according to any one of claims 29 to 32 wherein peptides of SEQ ID Nos. 1, 2, 3 and 4 are administered to said subject.

34. The method according to any one of claims 29 to 31 wherein said peptide is selected from SEQ ID Nos. 7, 8, 9 and 10.

35. The method according to any one of claims 29 to 31 and 34 wherein peptides of SEQ ID Nos. 7, 8, 9 and 10 are administered to said subject.

36. The method according to any one of claims 29 to 31 wherein said peptide is selected from SEQ ID Nos. 12, 16, 18, 23, 24, 25, 26, 27, 28, 29, 30 and 31.

37. A peptide as defined in any one of claims 32 to 36 for use in treating or preventing demyelination in a subject.

38. Use of a peptide as defined in any one of claims 32 to 36 in the manufacture of a medicament for treating or preventing demyelination in a subject.

39. The peptide for use according to claim 37 or use of a peptide according to claim 38 wherein said subject has a neurodegenerative disease.

40. The peptide for use according to claim 37 or use of a peptide according to claim 38 wherein said subject has multiple sclerosis, Alzheimer's disease, or Parkinson's disease.

41. A kit for use in treating or preventing demyelination in a subject, wherein said kit comprises a peptide as defined in any one of claims 32 to 36.


SEQUENCE LISTING (31 sequences)

SEQ ID NO: 1 - protein, 15 aa, Artificial Sequence
  Myelin basic protein (MBP) peptide, MBP 30-44
PRHRDTGILDSIGRF

SEQ ID NO: 2 - protein, 17 aa, Artificial Sequence
  MBP peptide, MBP 83-99
ENPVVHFFKNIVTPRTP

SEQ ID NO: 3 - protein, 15 aa, Artificial Sequence
  MBP peptide, MBP 131-145
ASDYKSAHKGFKGVD

SEQ ID NO: 4 - protein, 15 aa, Artificial Sequence
  MBP peptide, MBP 140-154
GFKGVDAQGTLSKIF

SEQ ID NO: 5 - protein, 218 aa, Homo sapiens
GQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 6 - protein, 21 aa, Homo sapiens
YRPPFSRVVHLYRNGKDQDGD

SEQ ID NO: 7 - protein, 15 aa, Artificial Sequence
  Myelin oligodendrocyte glycoprotein (MOG) peptide, MOG 41-55
RPPFSRVVHLYRNGK

SEQ ID NO: 8 - protein, 15 aa, Artificial Sequence
  MOG peptide, MOG 43-57
PFSRVVHLYRNGKDQ

SEQ ID NO: 9 - protein, 15 aa, Artificial Sequence
  MOG peptide, MOG 44-58
FSRVVHLYRNGKDQD

SEQ ID NO: 10 - protein, 15 aa, Artificial Sequence
  MOG peptide,  MOG 45-59
SRVVHLYRNGKDQDG

SEQ ID NO: 11 - protein, 276 aa, Homo sapiens
GLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 12 - protein, 26 aa, Artificial Sequence
  Myelin proteolipid protein (PLP) peptide, PLP 36-61
HEALTGTEKLIETYFSKNYQDYEYLI

SEQ ID NO: 13 - protein, 32 aa, Artificial Sequence
  PLP peptide, PLP 88-119
EGFYTTGAVRQIFGDYKTTICGKGLSATVTGG

SEQ ID NO: 14 - protein, 32 aa, Artificial Sequence
  PLP peptide, PLP 104-135
KTTICGKGLSATVTGGQKGRGSRGQHQAHSLE

SEQ ID NO: 15 - protein, 32 aa, Artificial Sequence
  PLP peptide, PLP 119-150
GQKGRGSRGQHQAHSLERVCHCLGKWLGHPDK

SEQ ID NO: 16 - protein, 28 aa, Artificial Sequence
  PLP peptide, PLP 179-206
TWTTCQSIAFPSKTSASIGSLCADARMY

SEQ ID NO: 17 - protein, 28 aa, Artificial Sequence
  PLP peptide, PLP 192-219
TSASIGSLCADARMYGVLPWNAFPGKVC

SEQ ID NO: 18 - protein, 28 aa, Artificial Sequence
  PLP peptide, PLP 207-234
GVLPWNAFPGKVCGSNLLSICKTAEFQM

SEQ ID NO: 19 - protein, 17 aa, Artificial Sequence
  PLP peptide, PLP 260-276
ATYNFAVLKLMGRGTKF

SEQ ID NO: 20 - protein, 19 aa, Artificial Sequence
  PLP peptide, PLP 39-57
LTGTEKLIETYFSKNYQDY

SEQ ID NO: 21 - protein, 19 aa, Artificial Sequence
  PLP peptide, PLP 180-198
WTTCQSIAFPSKTSASIGS

SEQ ID NO: 22 - protein, 15 aa, Artificial Sequence
  PLP peptide, PLP 208-222
VLPWNAFPGKVCGSN

SEQ ID NO: 23 - protein, 15 aa, Artificial Sequence
  PLP peptide, PLP 39-53
LTGTEKLIETYFSKN

SEQ ID NO: 24 - protein, 15 aa, Artificial Sequence
  PLP peptide, PLP 42-56
TEKLIETYFSKNYQD

SEQ ID NO: 25 - protein, 15 aa, Artificial Sequence
  PLP peptide, PLP 43-57
EKLIETYFSKNYQDY

SEQ ID NO: 26 - protein, 15 aa, Artificial Sequence
  PLP peptide, PLP 180-194
WTTCQSIAFPSKTSA

SEQ ID NO: 27 - protein, 15 aa, Artificial Sequence
  PLP peptide, PLP 181-195
TTCQSIAFPSKTSAS

SEQ ID NO: 28 - protein, 15 aa, Artificial Sequence
  PLP peptide, PLP 182-196
TCQSIAFPSKTSASI

SEQ ID NO: 29 - protein, 15 aa, Artificial Sequence
  PLP peptide, PLP183-197
CQSIAFPSKTSASIG

SEQ ID NO: 30 - protein, 15 aa, Artificial Sequence
  PLP peptide, PLP 184-198
QSIAFPSKTSASIGS

SEQ ID NO: 31 - protein, 15 aa, Artificial Sequence
  PLP peptide, PLP 208-222
VLPWNAFPGKVCGSN